(19) United States
(12) Patent Application Publication
(10) Pub. No.: US 2026/0151345 A1
(43) Pub. Date: Jun. 4, 2026

(54) FAST-DISSOLVING DOSAGE FORMS AND USES THEREOF

(71) Applicant: Astrocyte Pharmaceuticals, Inc., Cambridge, MA (US)

(72) Inventors: Russell Birch Poe, Groton Long Point, CT (US); Leon Paul Grother, Swindon (GB)

(21) Appl. No.: 19/126,330

(22) PCT Filed: Nov. 3, 2023
(86) PCT No.: PCT/US2023/036734

§ 371 Date: May 1, 2025

Related U.S. Application Data
(60) Provisional application No. 63/422,279, filed on Nov. 3, 2022.
(60) Provisional application No. 63/521,540, filed on Jun. 16, 2023.

(51) Int. Cl. A61K 9/20; A61K 9/00; A61K 31/7076

(52) U.S. Cl. CPC A61K 9/2063; A61K 9/0056; A61K 9/2018; A61K 9/2095; A61K 31/7076

(57) ABSTRACT

The present invention relates to pharmaceutical compositions for oral administration of A₃R agonist compounds. The compositions are designed to disintegrate or disperse rapidly to deliver an effective dose of the compound for treating, ameliorating, or promoting recovery from certain conditions of the brain, central nervous system (CNS), or cardiovascular system such as a brain injury, a neurodegenerative condition, or cardiac ischemia.

### CROSS-REFERENCE TO RELATED APPLICATIONS

This application claims the benefit of United States Provisional Application Nos. 63/521,540, filed Jun. 16, 2023; and 63/422,279, filed Nov. 3, 2022; the entirety of each of which is incorporated herein by reference.

### STATEMENT OF GOVERNMENT SUPPORT

This invention was made with government support under Grant No. W81XWH-15-9-0001 awarded by the USAMRDC. The government has certain rights in the invention.

### FIELD OF THE INVENTION

The present invention relates to pharmaceutical compositions for oral administration of A₃R agonist compounds. The compositions are designed to disintegrate or disperse rapidly to deliver an effective dose of the compound for treating, ameliorating, or promoting recovery from certain conditions of the brain, central nervous system (CNS), or cardiovascular system such as a brain injury, a neurodegenerative condition, or cardiac ischemia.

### BACKGROUND OF THE INVENTION

Brain injuries are a distressingly common medical condition and one of the leading causes of morbidity and mortality worldwide. The brain is particularly susceptible to injury as neurons have a limited capacity to repair. When an individual is born, the brain already has essentially all the neurons it will have in life. Unlike other cells in the body, neurons stop reproducing shortly after birth. If these cells are injured or die, they are not replaced, often culminating in the disabling and largely irreversible degradation of a person's cognitive and sensorimotor capacity. Conditions that result in nerve cell death and damage range from traumatic brain injuries (TBIs), to ischemic episodes (e.g., stroke) and trauma, to degenerative disorders (e.g., Alzheimer's disease).

After identifying clinical candidates for treatment of these injuries and diseases, there is a need to discover viable formulations capable of delivering the candidates to the body. Many TBI, stroke, and neurodegenerative condition patients are not capable of swallowing a tablet or capsule. Clinical studies have shown that 23-52% of patients with Parkinson's disease have swallowing difficulties and many such patients tend to dribble. Fast-dispersing dosage forms provide a possible solution in that they will disintegrate rapidly in the mouth, thereby minimizing the above problem as large volumes of water will not be co-administered. It is therefore anticipated that such fast-dispersing dosage forms will be easier for patients to take and easier for caregivers to administer. Minitabs and mucoadhesive dosage forms provide other options. Pharmaceutical formulations require a trial-and-error approach and extensive study of the therapeutic candidate's properties, such as crystallization behavior, polymorphism, particle size, and solubility in pharmaceutically acceptable excipients and solvents.

There is urgent and compelling unmet medical need for more effective treatments for brain injuries, CNS injuries, heart and cardiovascular diseases, and related conditions, as well as promoting neurorestoration in patients having a neurodegenerative condition such as Alzheimer's. The present invention meets this need and provides other related advantages.

### SUMMARY OF THE INVENTION

In one aspect, the present invention provides a pharmaceutical composition, comprising:
(a) compound A:

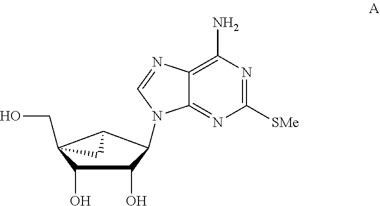

or a pharmaceutically acceptable salt thereof,
(b) a matrix former;
(c) a structure former; and, optionally,
(d) a flavoring agent and/or sweetening agent.

In some embodiments, the composition disintegrates in water at 20° C. in about 30 seconds or less.

The compounds may be administered via an oral disintegrating tablet.

### DETAILED DESCRIPTION OF THE INVENTION

### General Description of Certain Aspects of the Invention

The present invention provides pharmaceutical compositions for oral administration. As is well understood in the art, many active pharmaceutical agents (APIs) are not orally bioavailable. Even for APIs that are orally bioavailable, the amount of the API that is ultimately absorbed and distributed to the site(s) of action in the body is greatly influenced by the pharmaceutical formulation used. Discovering an optimal oral formulation is often challenging and unpredictable for various reasons, one of which is that the excipients interact both with each other and the API. A formulation that has been successful for one API will not necessarily be successful for a different API, even a structurally-similar one. In large part, formulation studies depend on trial and error for success and are not predictable in advance.

The present invention provides rapidly-disintegrating oral pharmaceutical compositions comprising an agonist of the adenosine receptor 1 (A₁R) and/or adenosine receptor 3 (A₃R). U.S. Pat. Nos. 9,789,131 and 10,765,693, the entirety of each of which is hereby incorporated herein by reference, describe certain therapeutically beneficial compounds. Such compounds include compound A:

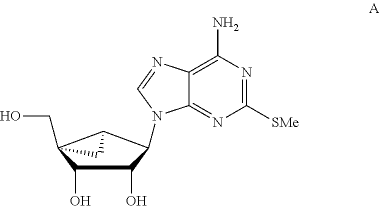

or a pharmaceutically acceptable salt thereof, as w ell as solid forms thereof.

Compound A has shown effectiveness in treatment of diseases such as TBI and stroke. See, e.g.:
1. Liston T E, Hama A, Boltze J, et al. Adenosine A1R/A3R (Adenosine A1 and A3 Receptor) Agonist AST-004 Reduces Brain Infarction in a Nonhuman Primate Model of Stroke. Stroke. 2022; 53(1):238-248. Epub 2021/11/23. doi: 10.1161/strokeaha.121.036396. PubMed PMID: 34802248.
2. Fisher E S, Chen Y, Sifuentes M M, et al. Adenosine A1R/A3R Agonist AST-004 Reduces Brain Infarction in Mouse and Rat Models of Acute Ischemic Stroke. Frontiers in Stroke (In Press). 2022.
3. Bozdemir E, Vigil F A, Chun S H, et al. Neuroprotective Roles of the Adenosine A(3) Receptor Agonist AST-004 in Mouse Model of Traumatic Brain Injury. Neurotherapeutics. 2021; 18(4):2707-2721. Epub 2021/10/06. doi: 10.1007/s13311-021-01113-7. PubMed PMID: 34608616; PMCID: PMC8804149.
4. Liston T E, Hinz S, Müller C E, et al. Nucleotide P2Y(1) receptor agonists are in vitro and in vivo prodrugs of A(1)/A(3) adenosine receptor agonists: implications for roles of P2Y(1) and A(1)/A(3) receptors in physiology and pathology. Purinergic Signal. 2020. Epub 2020/11/01. doi: 10.1007/s11302-020-09732-z. PubMed PMID: 33129204.
5. Suresh R R, Gao Z G, Salmaso V, et al. Selective A(3) Adenosine Receptor Antagonist Radioligand for Human and Rodent Species. ACS Med Chem Lett. 2022; 13(4):623-631. Epub 2022/04/23. doi: 10.1021/acsmedchemlett.1c00685. PubMed PMID: 35450351; PMCID: PMC9014498; Suresh R R, Poe R B, Lin B, et al. Convergent synthesis of 2-thioether-substituted (N)-methanocarba-adenosines as purine receptor agonists. RSC Adv. 2021; 11(44):27369-27380. Epub 2022/04/29. doi: 10.1039/dlra05096f. PubMed PMID: 35480676; PMCID: PMC9037833.

In one aspect, the present invention provides a pharmaceutical composition, comprising:
(a) compound A:

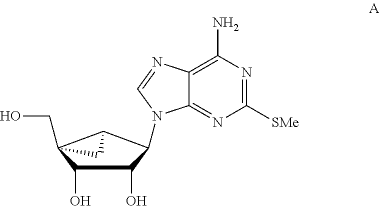

or a pharmaceutically acceptable salt thereof or solid form thereof;
(b) a matrix former;
(c) a structure former; and, optionally,
(d) a flavoring agent and/or a sweetening agent.

In one aspect, the present invention provides a pharmaceutical composition, comprising:
(a) compound A:

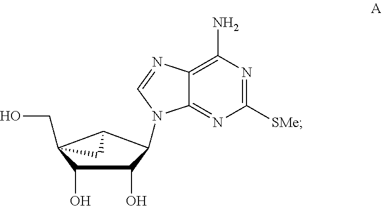

(b) a matrix former;
(c) a structure former; and, optionally,
(d) a flavoring agent and/or sweetening agent;
   wherein the composition disintegrates in water at 20° C. in about 30 seconds or less.

In some embodiments, the structure former comprises mannitol, PEG 400, hydrolyzed dextrose, dextran, dextrin, maltodextrin, an alginate, hydroxyethylcellulose, carboxymethylcellulose, microcrystalline cellulose, com-syrup solids, pectin, carrageenan, agar, chitosan, locust bean gum, xanthan gum, guar gum, acacia gum, tragacanth, konjac flour, rice flour, wheat gluten, sodium starch glycolate, soy fiber protein, potato protein, papain, glycine, sorbitol, erythritol, lactose, D-glucitol, trehalose, xylitol, or maltose; or a combination of two or more thereof.

In some embodiments, the composition comprises about 0.5% to about 60% water. In some embodiments, the composition comprises about 2% w/w to about 5%, about 10%, about 15%, about 20%, about 30%, about 40%, or about 50% w/w water. In some embodiments, the composition comprises about 2% to about 5% w/w water.

In some embodiments, the composition is prepared by lyophilization. In some embodiments, the composition is prepared by lyophilization of a pharmaceutical composition described above that is further comprising about 70% to about 85% w/w water in addition to compound A, about 0.5% to about 15% w/w of the matrix former, about 0.5% to about 15% w/w of the structure former, and optionally the sweetening agent and/or the flavoring agent. In some embodiments, the composition is prepared by lyophilization of a pharmaceutical composition described above comprising compound A as about 10% to about 15% N w/w of the composition; the matrix former comprises about 4.0% to about 6.0% w/w gelatin, and the structure former comprises about 3.0% to about 5.0% w/w mannitol. In some embodiments, compound A is present as about 12.8% w/w of the composition; the matrix former comprises about 5.0% w/w HMW fish gelatin; and the structure former comprises about 4.0% w/w mannitol.

In some embodiments, the structure former comprises mannitol.

In some embodiments, the mannitol is present as about 2.0% to about 60.0% w/w of the composition.

In some embodiments, the mannitol is present as about 10.0% to 24.0% w/w of the composition.

In some embodiments, the mannitol is present as about 16.0% w/w of the composition.

In some embodiments, the matrix former comprises a water-soluble or water-dispersible pharmaceutically acceptable carrier.

In some embodiments, the matrix former comprises gelatin, pullulan, starch, or combinations thereof.

In some embodiments, the matrix former comprises fish gelatin, bovine gelatin, porcine gelatin, or combinations thereof.

In some embodiments, the gelatin comprises Gel NonOx (Gelita), HMW (High Molecular Weight) bovine or HMW fish gelatin.

In some embodiments, the gelatin comprises HMW fish gelatin.

In some embodiments, the matrix former is present in an about 5:1 w/w to about 1:5 w/w ratio with the structure former.

In some embodiments, the matrix former is present in an about 1:1.25 w/w ratio with the structure former.

In some embodiments, the matrix former is present as about 2.0% to about 60.0% w/w of the composition.

In some embodiments, the matrix former is present as about 10.0% to about 40.0% w/w of the composition.

In some embodiments, the matrix former is present as about 14.0% to about 24.0% w/w of the composition.

In some embodiments, compound A is present as about 4% to about 90% w/w of the composition.

In some embodiments, compound A is about 20% to about 80% w/w of the composition.

In some embodiments, compound A is about 52% w/w of the composition.

In some embodiments, compound A is in the form of a hemi-hydrate.

In some embodiments, compound A comprises Form A having peaks in its XRPD at 8.0±0.2, 13.1±0.2, 16.2±0.2, 16.7±0.2, and 17.9±0.2 degree 2-theta.

In some embodiments, compound A is provided as a solid having D₉₀ of less than 30 μm prior to inclusion in the composition.

In some embodiments, the composition contains no more than 0.8% by HPLC, as compared to the AUC (area under the curve) of compound A, of the following compound:

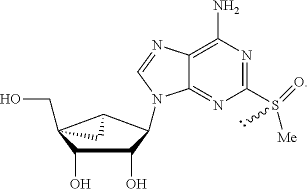

In some embodiments, the flavoring agent is present and is selected from coconut, orange, cherry, fruit punch, lemon, lime, grapefruit, raspberry, tutti-frutti, black cherry, strawberry, mint, peppermint, and combinations thereof, and the sweetening agent is present and is selected from sucrose, glucose, sucralose, fructose, lactose, aspartame, invert sugar, corn syrup, stevia extract powder, stevioside, steviol, saccharin, saccharin salts, potassium acetosulfam, sorbitol, xylitol, mannitol, erythritol, lactitol, alitame, miraculin, monellin, and thaumatin or a combination of the same.

In some embodiments, the composition comprises about 0.1% w/w sucralose and about 0.2% w/w peppermint.

In another aspect, the present invention provides a pharmaceutical composition comprising:
(a) compound A:

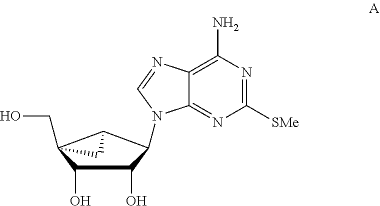

as about 4.0% to about 90.0% w/w of the composition;
(b) A matrix former comprising about 10.0% to about 40.0% w/w gelatin;
(c) A structure former comprising about 10.0% to about 24.0% mannitol; and, optionally,
(d) A flavoring agent and/or sweetening agent;
   wherein the composition disintegrates in water at 20° C. in about 30 seconds or less.

In some embodiments, compound A is present as about 10% to about 15% w/w of the composition; the matrix former comprises about 4.0% to about 6.0% w/w gelatin; and the structure former comprises about 3.0% to about 5.0% w/w mannitol.

In some embodiments, compound A is present as about 12.8% w/w of the composition: the matrix former comprises about 5.0% w/w HMW fish gelatin; and the structure former comprises about 4.0% w/w mannitol.

In some embodiments, the composition disintegrates in water at 20° C. in about 12 seconds or less.

In some embodiments, the composition disintegrates in water at 20° C. in about 7 seconds or less.

In some embodiments, the composition disintegrates in water at 20° C. in about 0.5 to about 5 seconds.

In another aspect, the present invention provides a unit dosage form comprising a pharmaceutical composition described herein, wherein the unit dosage form contains about 15 mg to about 1800 mg of compound A.

In some embodiments, the unit dosage form contains about 25 mg to about 1200 mg of compound A.

In some embodiments, the unit dosage form contains about 50 mg, about 150 mg, about 300 mg, or about 900 mg of compound A.

In some embodiments, the unit dosage form is an oral disintegrating tablet (ODT), granules, minitablet, mucoadhesive tablet, sublingual tablet, chewable tablet, or effervescent tablet.

In some embodiments, the unit dosage form is an oral disintegrating tablet (ODT).

In some embodiments, the unit dosage form comprises about 0.5% to about 60% water. In some embodiments, the unit dosage form comprises about 2% w/w to about 5%, about 10%, about 15%, about 20%, about 30%, about 40%, or about 50% w/w water. In some embodiments, the unit dosage form comprises about 2% to about 5% w/w water.

In some embodiments, the unit dosage form is prepared by lyophilization. In some embodiments, the unit dosage form is prepared by lyophilization of a pharmaceutical composition described above that is further comprising about 70% to about 85% w/w water in addition to compound A, about 0.5% to about 15% w/w of the matrix former, about 0.5% to about 15% w/w of the structure former, and optionally the sweetening agent and/or the flavoring agent. In some embodiments, the unit dosage form is prepared by lyophilization of a pharmaceutical composition described above comprising compound A as about 10% to about 15% w/w of the composition; the matrix former comprises about 4.0% to about 6.0% w/w gelatin; and the structure former comprises about 3.0% to about 5.0% w/w mannitol. In some embodiments, compound A is present as about 12.8% w/w of the composition; the matrix former comprises about 5.0% w/w HMW fish gelatin; and the structure former comprises about 4.0% w/w mannitol.

In some embodiments, the present invention provides a method of preparing a pharmaceutical composition described above, comprising:
(i) mixing water, a matrix former, and a structure former to form a pre-mix:
(ii) optionally, modifying the pH of the pre-mix to a desired pH value;
(iii) optionally, heating the pre-mix to a temperature of about 20-70° C.;
(iv) optionally, adding a flavoring agent;
(v) adding compound A:

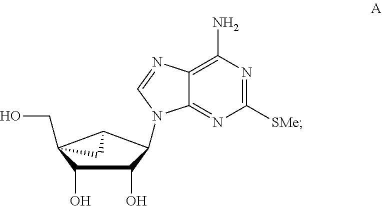

(vi) agitating the mix until compound A is dissolved, if compound A is not fully dissolved;
(vii) optionally, heating or cooling the mix of step (vi) to a temperature between about 10° C. and about 40° C.; and
(viii) removing the water and any volatile components.

In some embodiments, the present invention provides a method of preparing an oral disintegrating tablet (ODT), comprising:
(i) dosing a pharmaceutical composition into a preformed mold, wherein the pharmaceutical composition comprises a matrix former, a structure former, compound A:

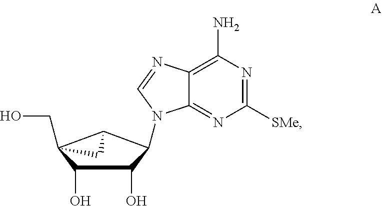

and, optionally, a flavoring agent and/or sweetening agent;
(ii) freezing the pharmaceutical composition; and
(iii) freeze-drying the pharmaceutical composition to form the ODT.

In some embodiments, compound A is provided in step (v) in the form of a hemi-hydrate.

In some embodiments, compound A comprises Form A having peaks in its XRPD at 8.0±0.2, 13.1±0.2, 16.2±0.2, 16.7±0.2, and 17.9±0.2 degree 2-theta.

In some embodiments, compound A is provided in anhydrous form, such as Form B described below.

In some embodiments, the present invention provides a method of treating an injury, disease, or condition selected from traumatic brain injury (TBI), concussion, stroke, polytrauma, cardiac arrest, near drowning, altitude sickness, brain injuries from directed energy or Havanna Syndrome, partial or total spinal cord transection, malnutrition, toxic neuropathies, meningoencephalopathies, neurodegeneration caused by a genetic disorder, age-related neurodegeneration, vascular disease, Alzheimer's Disease (AD), Parkinson's Disease (PD), Huntington's Disease (HD), Multiple Sclerosis (MS), amyotrophic lateral sclerosis (ALS), chronic traumatic encephalopathy (CTE), cardiovascular disease, autoimmune diseases, allergic diseases, transplant rejection, graft-versus-host disease, intraocular hypertension, glaucoma, odor sensitivity, an olfactory disorder, type 2 diabetes, pain control, respiratory diseases, deficits in CNS function, deficits in learning, deficits in cognition, otic disorders, Meniere's disease, endolymphatic hydrops, progressive hearing loss, noise-induced hearing loss, dizziness, vertigo, tinnitus, collateral brain damage associated with radiation cancer therapy, migraine treatment, sleep disorders in the elderly, epilepsy, schizophrenia, symptoms experienced by recovering alcoholics, damage to neurons or nerves of the peripheral nervous system during surgery, gastrointestinal conditions, pain mediated by the CNS, migraine, collateral brain damage associated with radiation cancer therapy, depression, mood or behavioral changes, dementia, erratic behavior, suicidality, tremors, Huntington's chorea, loss of coordination of movement, deafness, impaired speech, dry eyes, hypomimia, attention deficit, memory loss, cognitive difficulties, vertigo, dysarthria, dysphagia, ocular abnormalities or disorientation, and addiction; comprising administering to a patient in need thereof an effective amount of a composition described herein.

In some embodiments, the injury, disease, or condition is selected from acute pain, chronic pain, nociceptive pain, neuropathic pain, inflammatory pain, and acute pain.

In some embodiments, the pain is selected from musculoskeletal pain, fibromyalgia, myofascial pain, pain during menstruation, pain during osteoarthritis, pain during rheumatoid arthritis, pain during gastrointestinal inflammation, pain during inflammation of the heart muscle, pain during multiple sclerosis, pain during neuritis, pain during AIDS, pain during chemotherapy, tumor pain, headache, chronic pain syndrome (CPS), central pain, trigeminal neuralgia, shingles, stamp pain, phantom limb pain, temporomandibular joint disorder, nerve injury, migraine, post-herpetic neuralgia, neuropathic pain encountered as a consequence of injuries, amputation infections, metabolic disorders or degenerative diseases of the nervous system, neuropathic pain associated with diabetes, pseudesthesia, hypothyroidism, uremia, vitamin deficiencies or alcoholism, acute pain after injuries, postoperative pain, pain during acute gout, and pain from an operation.

In some embodiments, the injury, disease, or condition is selected from traumatic brain injury (TBI), stroke, a neurodegenerative condition, and a heart and cardiovascular disease.

In some embodiments, the injury, disease, or condition is TBI selected from concussion, blast injury, combat-related injury, a mild, moderate or severe blow to the head, whiplash, sports-related injury, or a head injury sustained from a fall or other accident.

In some embodiments, the injury, disease, or condition is a stroke selected from ischemic stroke, hemorrhagic stroke, subarachnoid hemorrhage, cerebral vasospasm, and transient ischemic attacks (TIA).

In some embodiments, the compound or composition is administered within 24 hours of the TBI or stroke.

In some embodiments, the compound or composition is administered within 8 hours of the TBI or stroke.

In some embodiments, the compound or composition is administered at least during the first 8-48 hours following the TBI or stroke.

In certain embodiments, the compound or composition is administered once an hour, every hour, for 1 to 8 hours. In certain embodiments, the compound or composition is administered once an hour, every hour, for 1 to 6 hours. In certain embodiments, the compound or composition is administered once an hour, every hour, for 1 to 4 hours. In certain embodiments, the compound or composition is administered once an hour, every hour, for 1 to 2 hours.

In some embodiments, neuroprotection or neurorestoration is increased in the patient as compared with an untreated patient.

In some embodiments, the injury, disease, or condition is a neurodegenerative disease selected from Alzheimer's Disease (AD), Parkinson's Disease (PD), Huntington's Disease (HD), Multiple Sclerosis (MS), amyotrophic lateral sclerosis (ALS), chronic traumatic encephalopathy (CTE), and a neurodegenerative condition caused by a virus, alcoholism, tumor, toxin, or repetitive brain injuries.

In some embodiments, the injury, disease, or condition is heart or cardiovascular disease selected from cardiac ischemia, myocardial infarction, a cardiomyopathy, coronary artery disease, arrhythmia, myocarditis, pericarditis, angina, hypertensive heart disease, endocarditis, rheumatic heart disease, congenital heart disease, and atherosclerosis.

In some embodiments, the present invention provides a method of increasing neuroprotection or neurorestoration in a patient in need thereof who has suffered a TBI or stroke, comprising administering to the patient an effective amount of a composition described herein.

In some embodiments, the present invention provides a method of treating an injury, disease, disorder, or condition selected from:
(i) brain damage caused by radiation or collateral brain damage associated with radiation cancer therapy or migraine treatment;
(ii) migraine headache;
(iii) a condition associated with a brain injury or a neurodegenerative condition; and
(iv) an autoimmune disease or condition, glaucoma, an otic disorder, progressive hearing loss, tinnitus, epilepsy, pain control, pain mediated by the CNS, neuropathic pain, inflammatory pain, or acute pain;
   comprising administering to a patient in need thereof an effective amount of a composition described herein.

In some embodiments, the composition increases neuroprotection or neurorestoration in the patient as compared with an untreated patient.

In some embodiments, the condition associated with a brain injury or a neurodegenerative condition is selected from epilepsy, migraine, collateral brain damage associated with radiation cancer therapy, depression, mood or behavioral changes, dementia, erratic behavior, suicidality, tremors, Huntington's chorea, loss of coordination of movement, deafness, impaired speech, dry eyes, hypomimia, attention deficit, memory loss, cognitive difficulties or deficit in cognition, deficit in CNS function, deficit in learning, vertigo, dysarthria, dysphagia, ocular abnormalities, and disorientation.

In some embodiments, the injury, disease, or condition is migraine.

In some embodiments, the injury, disease, or condition is pain selected from central pain syndrome, peripheral neuropathy, corneal neuropathic pain, post stroke pain, and pain caused by multiple sclerosis.

In some embodiments, the present invention provides a method of increasing cardioprotection or regeneration of damaged heart tissue in a patient in need thereof who has suffered a cardiac ischemia or myocardial infarction, comprising administering to the patient an effective amount of a composition described herein.

In some embodiments, the present invention provides a method of treating an addiction, addictive behavior, behavioral addiction, brain reward system disorder, or a compulsive disorder, comprising administering to a patient in need thereof an effective amount of a composition described herein.

In some embodiments, the present invention provides a method of treating a disease, disorder, or condition selected from deficit in cognition, deficit in CNS function, deficit in learning, and memory loss, comprising administering to a subject in need thereof an effective amount of a compound described herein, or a pharmaceutically acceptable salt thereof or a pharmaceutically acceptable composition thereof.

In some embodiments, the disease, disorder, or condition is deficit in cognition.

In some embodiments, the disease, disorder, or condition is deficit in CNS function.

In some embodiments, the disease, disorder, or condition is deficit in learning.

In some embodiments, the disease, disorder, or condition is memory loss.

In some embodiments, the subject has suffered one or more traumatic brain injuries (TBI or TBIs) and the disease, disorder, or condition is associated with the TBI or TBIs.

In some embodiments, the subject has suffered one or more strokes and the disease, disorder, or condition is associated with the one or more strokes.

In some embodiments, the subject has suffered one or more ischemic strokes, hemorrhagic strokes, subarachnoid hemorrhages, cerebral vasospasms, or transient ischemic attacks (TIA).

In some embodiments, the subject has Alzheimer's disease and the disease, disorder, or condition is associated with the Alzheimer's disease.

In some embodiments, a method provided herein improves cognitive or neurological function as measured by a score increase between about 1% and 40% in the delayed verbal recall task of the revised Wechsler Memory Scale.

In some embodiments, a method provided herein improves the score between about 5-10%, 10-20%, 15-30%, 20-30%, 30-40%, or 5-30% in the delayed verbal recall task of the revised Wechsler Memory Scale.

In some embodiments, the method increases synaptic plasticity, improves hippocampal long-term potentiation, improves cognitive function, decreases cognitive impairment, and/or improves or restores memory or learning.

In some embodiments, the method increases synaptic plasticity, improves hippocampal long-term potentiation, improves cognitive function, decreases cognitive impairment, prevents or delays cognitive decline, decreases plaque burden, enhances beta amyloid clearance, and/or improves or restores memory or learning.

In some embodiments, the method improves or enhances cognition or neurological function by enhancing synaptogenesis.

In one aspect, the present invention provides a method of improving cognitive or neurological function in a subject having Alzheimer's disease, comprising administering to a subject in need thereof an effective amount of Compound A, or a pharmaceutically acceptable salt thereof or a pharmaceutically acceptable composition thereof. In some embodiments, the improvement in cognitive or neurological function as measured by a score increase between about 1% and 40%, or about 5-10%, 10-20%, 15-30%, 20-30%, 30-40%, or 5-30%, in the delayed verbal recall task of the revised Wechsler Memory Scale.

The present invention provides a pharmaceutical composition for oral administration. In one embodiment, this composition is prepared from a slurry, suspension, or other mixture that comprises or consists essentially of water, mannitol, and gelatin at a concentration of up to 5% by weight as a carrier and compound A as an active pharmaceutical ingredient. In another embodiment, the dosage form according to the present invention is a solid, unitary, fast-dispersing dosage form that comprises a network of the active ingredient and a water-soluble or water dispersible carrier which is inert towards the active pharmaceutical ingredient. The network for the dosage form is obtained by subliming solvent from a composition in the solid state. That composition may consist essentially of the active ingredient and a solution of the carrier in a solvent. The dosage form according to the present invention will disintegrate completely within 1 to 30 seconds of being placed in the oral cavity.

Thus, the present invention provides a pharmaceutical composition for oral administration comprising or consisting essentially of gelatin at a concentration of up to 5% by weight as a carrier, water, and compound A, characterized in that the composition is in the form of a solid, unitary fast-dispersing dosage form consisting essentially of a network of the active pharmaceutical ingredient and a carrier which is inert towards the active pharmaceutical ingredient after subliming the water from the composition in the solid state. This dosage form completely disintegrates within 1 to 30 seconds of being placed in the oral cavity. In some embodiments, the pharmaceutical composition further comprises surfactants, preservatives, antioxidants, viscosity enhancers, coloring agents, flavoring agents, pH modifiers, sweeteners, and excipients.

There is also disclosed a solid unitary dosage form that disintegrates within 1 to 30 seconds of being placed in the oral cavity obtainable by the process of dispersing at least one matrix forming agent with a solvent to prepare a dispersion/solution and adding to said dispersion/solution compound A to prepare a dispersion/solution. Added to the dispersion solution is at least one agent selected from the group consisting of surfactants, preservatives, antioxidants, viscosity enhancers, coloring agents, flavoring agents, pH modifiers, sweeteners, and excipients to prepare a final dispersion. This final dispersion is then dispensed into preformed blister pockets and freeze dried in the blister pockets to obtain said unitary dosage form.

The term “fast-dispersing dosage form” encompasses all the types of dosage forms that are prepared by subliming a solvent from a composition or mixture that is in the solid state. However, it is preferred that the fast-dispersing dosage form comprises a network of the active ingredient and a water-soluble or water-dispersible carrier which is inert towards the active pharmaceutical ingredient, the network having been obtained by subliming solvent from a composition in the solid state, that composition comprising the active ingredient and a solution or dispersion of the carrier in a solvent.

In the case of the preferred type of fast-dispersing dosage form described above, the composition may contain, in addition to the active pharmaceutical ingredient, matrix forming agents or carriers and secondary components. Matrix forming agents or carriers suitable for use in the present invention include materials derived from animal or vegetable proteins, such as the gelatins, dextrins and soy, wheat and psyllium seed proteins; gums such as acacia, guar, agar, and xanthan: polysaccharides; alginates; carboxymethylcelluloses; carrageenans; dextrans; pectins; synthetic polymers such as polyvinylpyrrolidone; and polypeptide/protein or polysaccharide complexes such as gelatin-acacia complexes.

Other matrix forming agents or carriers suitable for use in the present invention include sugars such as mannitol, dextrose, lactose, galactose and trehalose: cyclic sugars such as cyclodextrin; inorganic salts such as sodium phosphate, sodium chloride and aluminum silicates; and amino acids having from 2 to 12 carbon atoms such as a glycine, L-alanine. L-aspartic acid, L-glutamic acid, L-hydroxyproline, L-isoleucine, L-leucine and L-phenylalanine.

One or more of these matrix forming agents can be combined to create a water-soluble or water dispersible carrier which is inert towards the active pharmaceutical ingredient.

One or more matrix forming agents may be incorporated into the solution or suspension prior to solidification. In addition to forming the matrix, the matrix forming agent or carrier may aid in maintaining the suspension of any active ingredient within the solution or suspension. This is especially helpful in the case of active agents that are not sufficiently soluble in water and must, therefore, be suspended rather than dissolved.

Secondary components that do not materially affect the basic and novel characteristics of the inventive composition and dosage form include materials such as preservatives, antioxidants, surfactants, viscosity enhancers, coloring agents, flavoring agents, pH modifiers, sweeteners or taste-masking agents. Suitable coloring agents include red, black and yellow iron oxides and FD & C dyes such as FD & C Blue No. 2 and FD & C Red No. 40 available from Ellis & Everard. Suitable flavoring agents include mint, raspberry, licorice, orange, lemon, grapefruit, caramel, vanilla, cherry and grape flavors and others described below. Suitable pH modifiers include citric acid, tartaric acid, phosphoric acid, hydrochloric acid and maleic acid. Suitable sweeteners include aspartame, acesulfame K and thaumatic and others described below. Suitable taste-masking agents include sodium bicarbonate, ion-exchange resins, cyclodextrin inclusion compounds, adsorbates or microencapsulated actives.

According to another aspect of the invention there is therefore provided a kit for co-administration of a composition containing a pharmaceutical composition previously defined. For instance, the kit may comprise at least one fast-dispersing unit dosage form according to the invention, optionally a pharmaceutical composition comprising a second therapeutic agent described herein, together with instructions for the administration of the unit dosage form(s).

### Forms of Compound a Present in Pharmaceutical Compositions of the Present Invention

The present invention provides pharmaceutical compositions comprising compound A or a pharmaceutically acceptable salt thereof. Compositions described herein include compound A or a pharmaceutically acceptable salt thereof in one or more of its physical forms. For example, compound A can be in solution, suspension, or in solid form. In certain embodiments, compound A is in solid form. When compound A is in solid form, said compound may be amorphous, crystalline, or a mixture thereof. Exemplary solid forms are described in more detail below.

In some embodiments, the present invention provides a form of compound A substantially free of impurities. As used herein, the term “substantially free of impurities” means that the compound contains no significant amount of extraneous matter. Such extraneous matter may include different forms of compound A, residual solvents, or any other impurities that may result from the preparation of, and/or isolation of, compound A. In certain embodiments, at least about 95% by weight of a form of compound A is present. In still other embodiments of the invention, at least about 99% by weight of a form of compound A is present.

According to one embodiment, a form of compound A is present in an amount of at least about 97, 97.5, 98.0, 98.5, 99, 99.5, 99.8 weight percent where the percentages are based on the total weight of the composition. According to another embodiment, a form of compound A contains no more than about 3.0 area percent HPLC of total organic impurities and, in certain embodiments, no more than about 1.5 area percent HPLC total organic impurities relative to the total area of the HPLC chromatogram. In other embodiments, a form of compound A contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound A is also meant to include all tautomeric forms of compound A. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹¹C-, ¹³C- or ¹⁴C-enriched carbon are within the scope of this invention.

It has been found that compound A can exist in a variety of solid forms. Exemplary such forms include polymorphs such as those described herein.

As used herein, the term “polymorph” refers to the different crystal structures into which a compound, or a salt or solvate thereof, can crystallize.

In certain embodiments, compound A is a crystalline solid. In other embodiments, compound A is a crystalline solid substantially free of amorphous compound A. As used herein, the term “substantially free of amorphous compound A” means that the compound contains no significant amount of amorphous compound A. In certain embodiments, at least about 95% by weight of crystalline compound A is present. In still other embodiments of the invention, at least about 99% by weight of crystalline compound A is present.

It has been found that compound A can exist in at least two distinct polymorphic forms. In certain embodiments, the present invention provides a polymorphic form of compound A referred to herein as Form A. In certain embodiments, the present invention provides a polymorphic form of compound A referred to herein as Form B.

In some embodiments, compound A is amorphous. In some embodiments, compound A is amorphous, and is substantially free of crystalline compound A.

### Form A of Compound A

In some embodiments, Form A of compound A has at least 1, 2, 3, 4 or 5 spectral peak(s) selected from the peaks listed in Table 1 below.

TABLE 1
XRPD Peak Positions for Form A of Compound A

| | Relative | | Relative | | Relative |
|---|---|---|---|---|---|
| °2θ¹ | Intensity | °2θ | Intensity | °2θ | Intensity |
| 7.6 | 1.49 | 21.5 | 35.63 | 30.8 | 12.79 |
| 8.0 | 100 | 22.8 | 2.30 | 32.0 | 8.32 |
| 9.0 | 3.49 | 23.1 | 6.16 | 32.8 | 8.27 |
| 10.8 | 4.60 | 23.7 | 4.21 | 32.9 | 13.11 |
| 11.8 | 1.59 | 23.9 | 9.39 | 33.7 | 9.73 |
| 12.5 | 4.59 | 24.9 | 54.39 | 34.7 | 4.31 |
| 13.1 | 59.02 | 26.1 | 32.89 | 36.3 | 3.98 |
| 16.2 | 34.56 | 26.5 | 13.26 | 36.7 | 8.82 |
| 16.7 | 37.68 | 26.6 | 22.22 | 37.9 | 12.68 |
| 17.2 | 10.65 | 27.1 | 60.62 | 38.2 | 3.16 |
| 17.9 | 45.59 | 28.6 | 10.52 | 38.5 | 1.75 |
| 18.1 | 16.05 | 29.3 | 1.66 | 38.7 | 2.83 |
| 18.3 | 10.91 | 29.7 | 9.05 | 39.6 | 2.37 |
| 19.8 | 4.96 | 30.1 | 1.89 | — | — |
| 21.0 | 30.62 | 30.4 | 1.77 | — | — |

¹In this and all subsequent tables, the position 2θ is within ± 0.2.

In some embodiments, Form A of compound A is characterized in that it has one or more peaks in its X-ray powder diffraction pattern selected from those at about 8.0 and about 13.1 degrees 2-theta. In some embodiments. Form A of compound A is characterized in that it has two peaks in its X-ray powder diffraction pattern selected from those at about 8.0 and about 13.1 degrees 2-theta. As used herein, the term “about,” when used in reference to a degree 2-theta value, refers to the stated value ±0.2 degree 2-theta.

Methods for preparing Form A of compound A are described infra.

### Form B of Compound A

In some embodiments, Form B of compound A has at least 1, 2, 3, 4 or 5 spectral peak(s) selected from the peaks listed in Table 2 below.

TABLE 2
XRPD Peak Positions for Form B of Compound A

| | Relative | | Relative | | Relative |
|---|---|---|---|---|---|
| °2θ¹ | Intensity | °2θ | Intensity | °2θ | Intensity |
| 4.7 | 2.43 | 20.1 | 88.35 | 29.5 | 4.27 |
| 7.6 | 3.96 | 21.0 | 42.70 | 29.9 | 3.19 |
| 9.5 | 14.73 | 21.5 | 68.69 | 30.2 | 6.20 |
| 10.0 | 10.55 | 23.0 | 2.13 | 30.6 | 7.95 |
| 10.5 | 7.20 | 23.8 | 53.95 | 31.6 | 4.20 |
| 13.8 | 20.73 | 24.3 | 8.09 | 32.3 | 1.42 |
| 14.2 | 10.53 | 24.6 | 3.80 | 32.7 | 5.03 |
| 14.7 | 40.39 | 25.4 | 5.74 | 33.1 | 4.67 |
| 15.2 | 6.59 | 25.6 | 8.51 | 33.6 | 3.56 |
| 15.4 | 14.70 | 25.9 | 35.31 | 35.9 | 7.11 |
| 16.2 | 4.80 | 26.2 | 20.76 | 37.0 | 2.87 |
| 17.1 | 24.58 | 26.6 | 16.53 | 37.4 | 1.78 |
| 17.9 | 58.03 | 27.6 | 7.29 | 39.0 | 1.26 |
| 18.3 | 12.94 | 28.8 | 25.11 | — | — |
| 19.0 | 100 | 29.1 | 8.40 | — | — |

¹In this and all subsequent tables, the position 2θ is within ± 0.2.

In some embodiments, Form B of compound A is characterized in that it has one or more peaks in its X-ray powder diffraction pattern selected from those at about 19.0, about 20.1 and about 21.5 degrees 2-theta. In some embodiments, Form B of compound A is characterized in that it has two or more peaks in its X-ray powder diffraction pattern selected from those at about 19.0, about 20.1 and about 21.5 degrees 2-theta. In some embodiments, Form B of compound A is characterized in that it has all three peaks in its X-ray powder diffraction pattern selected from those at about 19.0, about 20.1 and about 21.5 degrees 2-theta.

In some embodiments, Form B of compound A is characterized in that it has one or more peaks in its X-ray powder diffraction pattern selected from those at about 9.5, about 10.5 and about 13.8 degrees 2-theta. In some embodiments. Form B of compound A is characterized in that it has two or more peaks in its X-ray powder diffraction pattern selected from those at about 9.5, about 10.5 and about 13.8 degrees 2-theta. In some embodiments, Form B of compound A is characterized in that it has all three peaks in its X-ray powder diffraction pattern selected from those at about 9.5, about 10.5 and about 13.8 degrees 2-theta.

Methods for preparing Form B of compound A are described infra.

In some embodiments, the present invention provides compound A in crystalline form for use in a disclosed pharmaceutical composition.

In some embodiments, the present invention provides a solid form of compound A, wherein said compound is substantially free of amorphous compound A.

In some embodiments, the present invention provides a solid form of compound A, wherein said compound is substantially free of impurities.

In some embodiments, the present invention provides a solid form of compound A, wherein said compound has one or more peaks in its XRPD selected from those at about 8.0 and about 13.1 degrees 2-theta. In some such embodiments, the present invention provides compound 1, wherein said compound has two peaks in its XRPD selected from those at about about 8.0 and about 13.1 degrees 2-theta. In some such embodiments, the present invention provides Compound A, wherein said compound is of Form A.

In some embodiments, the present invention provides a solid form of compound A, wherein said compound has one or more peaks in its XRPD selected from those at about 19.0, about 20.1 and about 21.5 degrees 2-theta. In some such embodiments, the present invention provides compound A, wherein said compound has at least two peaks in its XRPD selected from those at about 19.0, about 20.1 and about 21.5 degrees 2-theta. In some embodiments, the present invention provides a solid form of compound A, wherein said compound has one or more peaks in its XRPD selected from those at about 9.5, about 10.5 and about 13.8 degrees 2-theta. In some such embodiments, the present invention provides compound A, wherein said compound has at least two peaks in its XRPD selected from those at about 0.5, about 10.5 and about 13.8 degrees 2-theta.

In some such embodiments, the present invention provides compound A, wherein said compound is of Form B.

In some embodiments, the compound or pharmaceutically acceptable salt thereof is administered orally, intravenously, or parenterally. In some embodiments, the compound or pharmaceutically acceptable salt thereof is administered orally. In some embodiments, the compound or pharmaceutically acceptable salt thereof is administered intravenously. In some embodiments, the compound or pharmaceutically acceptable salt thereof is administered parenterally. In some embodiments, the compound or pharmaceutically acceptable salt thereof is administered as a continuous intravenous (IV) infusion. In some embodiments, the compound or pharmaceutically acceptable salt thereof is administered initially as an IV bolus, followed by continuous IV infusion, e.g., to maintain a desired plasma concentration. In some embodiments, the compound or pharmaceutically acceptable salt thereof is administered as a slow bolus/rapid infusion. In some embodiments, the slow bolus/rapid infusion comprises IV administration over an about 5-60 minute period, e.g., a 5-30, 5-20, 10-20, or about 10 minute period.

### Uses of Compounds and Pharmaceutically Acceptable Compositions Thereof

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors).

### Brain, CNS, Cardiovascular, and Other Injuries and Conditions

In one aspect, the present invention provides a method of preventing and/or treating brain damage associated with acute brain trauma as well as longer term diseases of the brain and CNS and heart and cardiovascular diseases and conditions. In one aspect, the present invention provides methods of treating such injuries, diseases, and conditions by utilizing neuroprotective and neurorestorative effects mediated by astrocytes, which are now understood as the key natural caretaker cell of neurons, as well as the astrocyte mitochondria, which supply a significant portion of the brain's energy. In another aspect, the present invention provides methods of treating such injuries, diseases, and conditions by cardioprotective and regenerative effects mediated by A₃R receptors. Regarding neuroprotective and neurorestorative effects, without wishing to be bound by theory, it is believed that selective enhancement of astrocyte energy metabolism mediated by A₃R and/or P2Y₁ receptors promotes astrocyte caretaker functions, such as their neuroprotective and neurorestorative functions, in turn enhancing the resistance of neurons and other cells to both acute injury and long-term stress. In some cases, it may be advantageous to achieve biased. i.e., selective or preferential, of one or more pathways mediated by A₃R and/or P2Y₁ and/or A₁R receptors wherein one or more undesired pathways are not activated or activated to a lesser degree. In addition to or as an alternative to astrocytes, neuroprotective or neurorestorative function of glia, microglia, neurons, endothelium cells and other brain and/or CN S cell types may be activated. Accordingly, in one aspect, the present invention provides compounds and methods of use thereof for treating, ameliorating, or promoting recovery from certain conditions of the brain or central nervous system (CNS) such as brain injuries, for example by increasing neuroprotection and/or neurorestorative effects mediated by astrocytes, glia, microglia, neurons, endothelium cells or other cells of the brain and/or CNS, comprising administering to a patient in need thereof an effective amount of a compound described herein, or a pharmaceutically acceptable salt thereof or composition comprising the same.

Astrocytes play key roles in supporting and protecting neurons and they critically affect the outcome of brain injuries that cause brain damage, such as ischemic injuries. The central role astrocyte mitochondria themselves play in these brain functions is less well appreciated. For example, inhibition of astrocyte mitochondria increases swelling and leads to necrotic cell death. Neurons are permanently injured by recurrent spreading depolarizations only if astrocyte mitochondrial function fails, and astrocyte mitochondria are required for reduction of pathophysiological elevations of extracellular K⁺, which initiate spreading depolarizations. Activation of purinergic receptors on astrocytes results in increased mitochondrial Ca²⁺ that enhances mitochondrial citric acid cycle function and increases respiration and ATP production. Accordingly, in one aspect, the present invention relates to the discovery that activation of astrocyte purinergic receptors enhances brain cell survival signalling pathways, enabling both astrocyte and neuronal viability during oxidative stress. Furthermore, activated astrocytes generate and supply reduced glutathione, a key antioxidant that aids in the resistance of both astrocytes and neurons to oxidative stress. Thus, in one aspect, the present invention provides a method of modulating astrocyte purinergic receptors to promote survival and viability of one or more cell types in the brain of a patient after oxidative stress, such as oxidative stress caused by a brain injury, ischemia-reperfusion or a neurodegenerative condition, comprising administering to a patient in need thereof a compound described herein, or a pharmaceutically acceptable salt thereof or composition comprising the same.

In some embodiments, activation of astrocytes is achieved through contacting with a disclosed compound one or more purinergic receptors such as adenosine receptors (Ars), for example those associated with or expressed by astrocytes, thus modulating the activity of the one or more receptors. In some embodiments, through effects on adenosine receptors such as A₁, A_{2A}, A_{2B} and A₃ on astrocytes, the compound activates astrocytes to treat one or more disclosed diseases or conditions. In some embodiments, after administration to a patient in need thereof, a disclosed compound influences one or more astrocyte functions. In some embodiments, the astrocyte function is selected from glutamate uptake, reactive gliosis, swelling, or release of neurotrophic and neurotoxic factors that act to ameliorate metabolic stress and its consequences. In some embodiments, the compound is an AR agonist. In some embodiments, the purinergic receptor is an A₃ adenosine receptor (A₃R). In some embodiments, the compound is an A₃R agonist. In some embodiments, the compound is a partial agonist or biased agonist or biased partial agonist, at an A₃ receptor (A₃R), such as a human A₃ receptor (hA₃R). In some embodiments, the compound acts as an agonist of an A₁ adenosine receptor (A₁R). In some embodiments, the compound is a biased agonist at an A₁ and/or A₃ receptor. In some embodiments, the compound acts by dual agonism at an A₃R and an A₁R.

In another aspect, the present invention provides a method of treating or ameliorating a brain injury, disease, or condition, such as a brain injury resulting from a TBI or progressive neurodegenerative disorder, in a patient in need thereof, comprising administering to the patient an effective amount of a compound described herein, or a pharmaceutically acceptable salt thereof or composition comprising the same. In some embodiments, the subject has suffered a TBI, concussion, stroke, partial or total spinal cord transection, or malnutrition. In other embodiments, the subject has suffered toxic neuropathies, meningoencephalopathies, neurodegeneration caused by a genetic disorder, age-related neurodegeneration, or a vascular disease; or another disease disclosed in U.S. Pat. No. 8,691,775, which is hereby incorporated by reference. In some embodiments, the present invention provides a method of treating or ameliorating a brain injury, disease, or condition, such as a brain injury resulting from a TBI or progressive neurodegenerative disorder, in a patient in need thereof, comprising administering to the patient an effective amount of a compound described herein, or a pharmaceutically acceptable salt thereof or composition comprising the same, wherein the compound is an A₁R and/or A₃R agonist. In some embodiments, the compound is a biased agonist, partial agonist, or biased partial agonist at an A₁ receptor. In some embodiments, the compound is a biased agonist, partial agonist, or biased partial agonist at an A₃ receptor. In some embodiments, the compound acts by dual agonism at an A₃R and an A₁R.

In another aspect, the present invention provides a method of promoting or increasing neuroprotection, neurorestoration, or neuroregeneration in a patient suffering from a disease or condition, comprising administering to the patient an effective amount of a compound described herein, or a pharmaceutically acceptable salt thereof or composition comprising the same. In some embodiments, the patient is suffering from a neurodegenerative disease or condition. In some embodiments, the patient has suffered a TBI or stroke.

### Traumatic Brain Injuries

Traumatic brain injuries (TBI) are a distressingly common medical condition. There are no approved treatments for TBI, and most TBI patients are discharged from the hospital with no pharmacological treatment. Repetitive TBI such as concussions can trigger age-associated neurodegeneration that results in a range of symptoms and disabilities over decades. TBIs can happen through sports-related injuries, motor vehicle accidents, falls, explosive impacts, physical assaults, etc. Injuries range widely in their complexity and severity, from “mild” concussions with brief alterations in mental status, cognitive difficulties, or loss of consciousness to “severe” with prolonged periods of unconsciousness and/or amnesia after the injury. In the U.S., approximately 1.7 million people have an injury resulting in a TBI annually and seek medical intervention (USCSF and CDC), and the CDC estimates that 1.6 to 3.8 million additional concussion incidents occur in sports and other recreational pursuits annually that do not present to hospital or emergency departments. Approximately 5-10% of athletes will receive a concussion each sport season. Football is the sport with the highest concussion risk for males (75% chance for concussion), while soccer has the highest concussion risk for females (50% chance for concussion). TBI is the leading cause of death and disability in children and young adults (CDC) and the most commonly received military-related injury; approximately 20% of U.S. Service Members deployed since 2003 have sustained at least one TBI. Total TBI-related indirect and direct medical costs are estimated at $77 billion annually. At least 5 million Americans require ongoing daily support in performing activities as a result of TBI.

Provided herein in one aspect is a method of treating TBI or promoting recovery from TBI, comprising administering to a patient in need thereof an effective amount of a compound described herein, or a pharmaceutically acceptable salt thereof or composition comprising the same. In some embodiments, the TBI is selected from traumatic injuries to the brain (such as concussion, blast injury, combat-related injury) or spinal cord (such as partial or total spinal cord transection). In some embodiments, the TBI results from a mild, moderate, or severe blow to the head, comprises an open or closed head wound, or results from a penetrating or non-penetrating blow to the head. In some embodiments, the TBI is selected from concussion, blast injury, combat-related injury, a mild, moderate or severe blow to the head, whiplash, sports-related injury, or a head injury sustained from a fall or other accident.

### Stroke

A stroke occurs when a blood vessel that transports oxygen and nutrients to the brain is disrupted due to an ischemic blockage or from the hemorrhagic rupture of a blood vessel in the brain, causing neurons, glia and endothelial cells in the disrupted region of the brain to die. The outcome of the stroke depends upon the location and breadth of damage, and the impacts of that damage are observed in the body functions regulated by the damaged brain region. Strokes can cause unilateral or bilateral paralysis, speech and language disabilities, memory loss, behavioral changes, and even death. Stroke is the fourth leading cause of death in the United States and is a major cause of adult disability. Each year, ˜800,000 people experience a new or recurrent stroke. Each day, over 2,000 Americans will have a stroke, resulting in death in over 400 of these incidents. Stroke accounted for ˜1 of every 19 deaths in the United States in 2010. An estimated 6.8 million Americans ≥20 years of age have had a stroke. As of 2010, the annual direct and indirect cost of stroke was estimated at $36.5 billion. Within minutes of a stroke, the lack of blood flow will permanently damage a core of brain tissue. Between this damaged core and normal brain tissue is a region of tissue known as the penumbra—tissue that is under gradated stress from lessened blood flow and some disruption of energy metabolism. Over the first 24-48 hours following a stroke incident, the stress on neuronal and glia cells in the penumbra resolves either with some recovery or further cell death.

In one aspect, the present invention provides a method of neuroprotective therapy in a stroke patient, comprising administering to a patient in need thereof an effective amount of a compound described herein, or a pharmaceutically acceptable salt thereof or composition comprising the same. In some embodiments, such therapy salvages as much of the penumbra as possible, and/or limits further acute tissue damage, and/or promotes neuron recovery. In another aspect is provided a method of treating stroke or promoting recovery from stroke, comprising administering to a patient in need thereof an effective amount of a compound described herein, or a pharmaceutically acceptable salt thereof or composition comprising the same. In another aspect is provided a method of promoting or increasing neuroprotection, neuroregeneration, or neurorestoration in a patient who has suffered a stroke, comprising administering to the patient an effective amount of a compound described herein, or a pharmaceutically acceptable salt thereof or composition comprising the same.

In some embodiments, the stroke is selected from ischemic stroke, hemorrhagic stroke, subarachnoid hemorrhage, cerebral vasospasm, and transient ischemic attacks (TIA). In some embodiments, the stroke is ischemic, e.g., an acute ischemic stroke (AIS). In some embodiments, the stroke is hemorrhagic. In some embodiments, the compound is administered within 48 hours of the stroke. In some embodiments, the compound is administered within 24 hours of the stroke. In some embodiments, the compound is administered within 16 hours of the stroke. In some embodiments, the compound is administered within 8, 4, 2, or 1 hours of the stroke. In some embodiments, the compound is administered for at least the first 1-72 hours following the stroke. In some embodiments, the compound is administered for at least the first 8-52 hours following the stroke. In some embodiments, the compound is administered for at least the first 8-48 hours following the stroke. In some embodiments, the compound is administered for at least the first 24-48 hours following the stroke. In some embodiments, the compound is administered chronically to treat the stroke as it occurs. In some embodiments, the compound is administered chronically to treat Transient Ischemic Attacks (TIA).

In some embodiments, the compound is administered chronically to treat ischemic stroke, hemorrhagic stroke, a subarachnoid hemorrhage, cerebral vasospasm, transient ischemic attacks (TIA), or treat a patient who is at an increased risk for a stroke, such as a patient who has had a stroke in the past and is at risk for a further stroke, such as a patient over the age of 40, 45, 50, 55, 60, 65, 70, 75, or 80 years of age.

In some embodiments, the compound treats an ischemia-reperfusion injury caused by the stroke.

In certain embodiments, for treatment of stroke and related conditions, a recanalization procedure such as thrombolysis by recombinant tissue plasminogen activator (r-tPA) or mechanical thrombectomy is used in combination with a presently disclosed method of treating stroke or the related condition.

### Neurodegenerative Diseases

Neurodegenerative diseases are incurable, progressive, and ultimately debilitating syndromes resulting from the progressive degeneration and/or death of neurons in the brain and spinal cord. Neurodegeneration results in movement (ataxias) and/or cognitive function (dementias) disorders, and includes a spectrum of diseases such as Alzheimer's Disease (AD), Parkinson's Disease (PD), Huntington's Disease (HD), Multiple Sclerosis (MS), amyotrophic lateral sclerosis (ALS), and chronic traumatic encephalopathy (CTE). While many neurodegenerative diseases are principally genetic in origin, other causes can include viruses, alcoholism, tumors or toxins, and as is now clear, repetitive brain injuries.

Neurons accumulate cellular damage over time due to the foregoing factors, which is generally considered the reason why many neurodegenerative diseases associated with prolonged cellular stress, such as Alzheimer's disease and Parkinson's disease, occur in aged individuals. Dementias represent the predominant outcome of neurodegenerative diseases with AD representing approximately 60-70% of cases. As discussed above, activation of neuroprotective and neurorestorative mechanisms can ameliorate the progression of one or more neurodegenerative diseases. Accordingly, in one aspect the present invention provides a method of treating a neurodegenerative disease or promoting recovery from a neurodegenerative disease, comprising administering to a patient in need thereof an effective amount of a compound described herein, or a pharmaceutically acceptable salt thereof or composition comprising the same.

In one aspect, the present invention provides a method of promoting neuroprotection or neurorestoration in a patient suffering from a neurodegenerative disease, comprising administering to the patient an effective amount of a compound described herein, or a pharmaceutically acceptable salt thereof or composition comprising the same.

### Alzheimer's Disease (AD)

An estimated 5.2 million Americans of all ages had AD in 2014; 11% of the population age 65 and older have AD. By 2050, the number of people age 65 and older with AD is projected to nearly triple to a projected 13.8 million. In the U.S., the cost of providing care for AD patients is about $214 billion per year: 70% of this cost is covered by Medicare and Medicaid. The current trends would project these costs to grow to $1.2 trillion per year by 2050.

Activation of astrocytes and promoting neuroprotection and neurorestoration according to the present invention represents a new treatment option for AD. Accordingly, provided herein in one aspect is a method of treating AD or promoting neuroprotection or neurorestoration in a patient suffering from AD, comprising administering to the patient an effective amount of a compound described herein, or a pharmaceutically acceptable salt thereof or composition comprising the same.

In some embodiments, beneficial effects resulting from a method of treating AD provided herein include, but are not limited to, one or more of, improving cognitive function, decreasing cognitive impairment, decreasing plaque burden, enhancing beta amyloid clearance, increasing synaptogenesis, and improving memory.

### Parkinson's Disease (PD)

As many as one million Americans live with PD, and each year approximately 60,000 Americans are newly diagnosed not including the thousands of cases that go undetected. The total combined direct and indirect cost of PD, including medical treatment, social security payments and lost income, is estimated to be nearly $25 billion per year in the United States.

Activation of neuroprotection and neurorestoration according to the present invention represents a new treatment option for PD. Accordingly, provided herein in one aspect is a method of treating PD or promoting neuroprotection or neurorestoration in a patient suffering from PD, comprising administering to the patient an effective amount of a compound described herein, or a pharmaceutically acceptable salt thereof or composition comprising the same.

### Multiple Sclerosis (MS)

More than 400,000 people in the United States have MS. In young adults, MS represents the most prevalent disease of the central nervous system. There is potential for astrocytes to reverse the destruction of nerve cell myelin coatings that is caused by MS by their neurorestorative effects and promotion of healing in the damaged CNS of MS patients.

Activation of neuroprotection and neurorestoration in the CNS according to the present invention thus represents a new treatment option for MS. Accordingly, provided herein in one aspect is a method of treating MS or promoting neuroprotection or neurorestoration in a patient suffering from MS, comprising administering to the patient an effective amount of a compound described herein, or a pharmaceutically acceptable salt thereof or composition comprising the same.

### Amyotrophic Lateral Sclerosis (ALS)/Lou Gehrig's Disease

Approximately 5,600 people in the U.S. are diagnosed with ALS each year; as many as 30,000 Americans may have the disease concurrently. Activation of astrocytes can provide stimulation of recovery and repair of the neurons and their connections in an ALS patient.

Accordingly, provided herein in one aspect is a method of treating ALS or promoting neuroprotection or neurorestoration in a patient suffering from ALS, comprising administering to the patient an effective amount of a compound described herein, or a pharmaceutically acceptable salt thereof or composition comprising the same. Also provided in other embodiments is a method of stimulating recovery and repair of the neurons and their connections in an ALS patient, comprising administering to the patient an effective amount of a compound described herein, or a pharmaceutically acceptable salt thereof or composition comprising the same.

### Chronic Traumatic Encephalopathy (CTE)

CTE (a form of tauopathy) is a progressive neurodegenerative disease found in individuals who have suffered one or more (often multiple or repeated over the course of time) severe blows to the head. CTE is most often diagnosed in professional athletes in American football, soccer, hockey, professional wrestling, stunt performing, bull riding and rodeo performing, motocross, and other contact sports who have experienced brain trauma and/or repeated concussions. A subset of CTE sufferers have chronic traumatic encephalomyopathy (CTEM), which is characterized by motor neuron disease symptoms that mimic ALS. Progressive muscle weakness and motor and gait abnormalities are believed to be early signs of CTEM. First stage symptoms of CTE include progressive attention deficit, disorientation, dizziness, and headaches. Second stage symptoms comprise memory loss, social instability, erratic behavior, and poor judgment. In third and fourth stages, patients suffer progressive dementia, slowed movements, tremors, hypomimia, vertigo, speech impediments, hearing loss, and suicidality, and may further include dysarthria, dysphagia, and ocular abnormalities, e.g., ptosis.

Accordingly, provided herein in one aspect is a method of treating or preventing CTE or promoting neuroprotection or neurorestoration in a patient suffering from CTE, comprising administering to the patient an effective amount of a compound described herein, or a pharmaceutically acceptable salt thereof or composition comprising the same. Also provided in other embodiments is a method of stimulating recovery and repair of the neurons and their connections in a CTE patient, comprising administering to the patient an effective amount of a compound described herein, or a pharmaceutically acceptable salt thereof or composition comprising the same. In some embodiments, the compound treats one or more symptoms of first stage, second stage, third stage, or fourth stage CTE.

On a microscopic scale the pathology includes neuronal death, tau deposition, TAR DNA-binding Protein 43 (TDP 43) beta-amyloid deposition, white matter changes, and other abnormalities. Tau deposition includes the increasing presence of dense neurofibrillary tangles (NFT), neurites, and glial tangles, which are made up of astrocytes and other glial cells. Thus, in some embodiments, the method treats, enhances clearance or prevents neuronal death, tau deposition, TAR DNA-binding Protein 43 (TDP 43) beta-amyloid deposition, white matter changes, and other abnormalities associated with CTE.

### Cardiovascular Diseases

Disclosed compounds are also useful in treating a variety of cardiovascular diseases and conditions. In some embodiments, the present invention provides a method of treating a heart (cardiac) or cardiovascular disease, such as cardiac ischemia, myocardial infarction, a cardiomyopathy, chest pain, stress, coronary artery disease, arrhythmia, myocarditis, pericarditis, angina, hypertensive heart disease, endocarditis, rheumatic heart disease, congenital heart disease, or atherosclerosis, comprising administering an effective amount of a compound described herein, or a pharmaceutically acceptable salt thereof or composition comprising the same.

In some embodiments, the heart or cardiovascular disease is cardiac ischemia or myocardial infarction.

In some embodiments, the present invention provides a method of promoting or increasing cardioprotection, cardiorestoration, or cardioregeneration in a patient suffering from a heart (cardiac) or cardiovascular disease or condition, comprising administering to the patient an effective amount of a disclosed compound, or a pharmaceutically acceptable salt thereof or a composition comprising the same.

In some embodiments, the heart (cardiac) or cardiovascular disease from which the patient is suffering is cardiac ischemia, myocardial infarction, a cardiomyopathy, coronary artery disease, arrhythmia, myocarditis, pericarditis, angina, hypertensive heart disease, endocarditis, rheumatic heart disease, congenital heart disease, or atherosclerosis.

### Other Diseases

Compounds that modulate beneficial effects such as neuroprotection, for example by increasing astrocyte mitochondrial activity, also have the potential to treat a variety of other diseases. For example, due to the role of astrocytes in neuroprotection disclosed in the present invention, activation of astrocytes, for example via modulation of A₃R and/or A₁R, is useful in treating various diseases and conditions discussed below.

Accordingly, in some embodiments, the present invention provides a method of treating neurodegeneration in a patient suffering from a disease or condition, comprising administering to the patient an effective amount of a compound described herein, or a pharmaceutically acceptable salt thereof or composition comprising the same.

In some embodiments, the present invention provides a method of promoting or increasing neuroprotection, neurorestoration, or neuroregeneration in a patient suffering from a disease or condition, comprising administering to the patient an effective amount of a compound described herein, or a pharmaceutically acceptable salt thereof or composition comprising the same.

In some embodiments, the disease or condition is selected from autoimmune diseases, allergic diseases, and/or transplant rejection and graft-versus-host disease (for the use of certain nucleoside and nucleotide compounds in treating these conditions, see, for example, WO 2007/20018, hereby incorporated by reference). In other embodiments, the disease or condition is selected from intraocular hypertension and/or glaucoma (for the use of certain nucleoside and nucleotide compounds in treating these conditions, see, for example, WO 2011/77435, hereby incorporated by reference). In other embodiments, the disease or condition is selected from odor sensitivity and/or an olfactory disorder (for the use of certain nucleoside and nucleotide compounds in treating these conditions, see, for example, EP1624753, hereby incorporated by reference). In other embodiments, the disease or condition is type 2 diabetes (for the use of certain nucleoside and nucleotide compounds in treating these conditions, see, for example, US 2010/0256086, hereby incorporated by reference).

In other embodiments, the disease or condition is selected from respiratory diseases and/or cardiovascular (CV) diseases (for the use of certain nucleoside and nucleotide compounds in treating these conditions, see, for example, FASEB J. (2013) 27:1118.4 (abstract of meeting), hereby incorporated by reference). In other embodiments, the disease or condition is selected from deficits in CNS function, deficits in learning and/or deficits in cognition (for the use of certain nucleoside and nucleotide compounds in treating these conditions, see, for example, Neuropsychopharmacology 2015 January; 40(2):305-14. Doi: 10.1038/npp.2014.173. Epub 2014 Jul. 15. “Impaired cognition after stimulation of a P2Y₁ receptor in the rat medial prefrontal cortex,” Koch, H. et al. PMID: 25027332, hereby incorporated by reference). In other embodiments, the disease or condition is selected from a neurodegenerative disease such as Alzheimer's disease, Parkinson's disease, Huntington's disease, prion disease, and/or amyotrophic lateral sclerosis (for the use of certain nucleoside and nucleotide compounds in treating these conditions see, for example, U.S. Pat. No. 8,691,775, hereby incorporated by reference). In other embodiments, the disease or condition is selected from otic disorders. Meniere's disease, endolymphatic hydrops, progressive hearing loss, noise-induced hearing loss, dizziness, vertigo, tinnitus, collateral brain damage associated with radiation cancer therapy, and/or migraine treatment (for the use of certain nucleoside and nucleotide compounds in treating these conditions, see, for example, US 2009/0306225; UY31779; and U.S. Pat. No. 8,399,018, each of which is hereby incorporated by reference). In other embodiments, the disease or condition is selected from pathological sleep perturbations, depression, sleep disorders in the elderly, Parkinson's disease, Alzheimer's disease, epilepsy, schizophrenia, and/or symptoms experienced by recovering alcoholics (for the use of certain nucleoside and nucleotide compounds in treating these conditions, see, for example, US 2014/0241990, hereby incorporated by reference). In other embodiments, the disease or condition is selected from damage to neurons or nerves of the peripheral nervous system during surgery (for the use of certain nucleoside and nucleotide compounds in treating these conditions, see, for example, U.S. Pat. No. 8,685,372, hereby incorporated by reference). In other embodiments, the disease or condition is a cancer such as prostate cancer (for the use of certain nucleoside and nucleotide compounds in treating these conditions, see, for example, Biochem Pharmacol. 2011 Aug. 15: 82(4): 418-425. Doi:10.1016/j.bcp.2011.05.013. “Activation of the P2Y1 Receptor Induces Apoptosis and Inhibits Proliferation of Prostate Cancer Cells,” Qiang Wei et al., hereby incorporated by reference). In other embodiments, the disease or condition is selected from one or more gastrointestinal conditions such as constipation and/or diarrhea (for the use of certain nucleoside and nucleotide compounds in treating these conditions, see, for example, Acta Physiol (Oxf). 2014 December; 212(4):293-305. Doi: 10.1111/apha.12408. “Differential functional role of purinergic and nitrergic inhibitory cotransmitters in human colonic relaxation,” Mañé N1, Gil V, Martinez-Cutillas M, Clavé P, Gallego D, Jimenez M.; and Neurogastroenterol. Motil. 2014 January; 26(1):115-23. Doi: 10.1111/nmo.12240. Epub 2013 Oct. 8. “Calcium responses in subserosal interstitial cells of the guinea-pig proximal colon,” Tamada H., Hashitani H. PMID: 24329947, hereby incorporated by reference).

In other embodiments, the disease or condition is selected from cancer of the brain, such as glioblastoma (for the use of certain nucleoside and nucleotide compounds in treating these conditions, see, for example, Purinergic Signal. 2015 September; 11(3):331-46. Doi: 10.1007/s11302-015-9454-7. Epub 2015 May 15. “Potentiation of temozolomide antitumor effect by purine receptor ligands able to restrain the in vitro growth of human glioblastoma stem cells.” D'Alimonte, I. et al. PMID: 25976165, hereby incorporated by reference). In other embodiments, the disease or condition is selected from a gastrointestinal disorder such as diarrhea (for the use of certain nucleoside and nucleotide compounds in treating these conditions, see, for example, Acta Physiol (Oxf). 2014 December; 212(4):293-305. Doi: 10.1111/apha.12408. “Differential functional role of purinergic and nitrergic inhibitory cotransmitters in human colonic relaxation,” Mañé N., Gil V, Martinez-Cutillas M, Clavé P, Gallego D, Jimenez M., hereby incorporated by reference). In other embodiments, the disease or condition is impaired cognition (for the use of certain nucleoside and nucleotide compounds in treating this condition, see, for example, Neuropsychopharmacology. 2015 January; 40(2):305-14. Doi: 10.1038/npp.2014.173. Epub 2014 Jul. 15. “Impaired cognition after stimulation of P2Y₁ receptors in the rat medial prefrontal cortex,” Koch H, Bespalov A, Drescher K, Franke H, Krügel U. PMID: 25027332, hereby incorporated by reference).

In some embodiments, the present invention provides a method of treating a disease or condition associated with brain injury or a neurodegenerative condition, such as epilepsy, migraine, collateral brain damage associated with radiation cancer therapy, depression, mood or behavioral changes, dementia, erratic behavior, suicidality, tremors, Huntington's chorea, loss of coordination of movement, deafness, impaired speech, dry eyes, hypomimia, attention deficit, memory loss, cognitive difficulties, vertigo, dysarthria, dysphagia, ocular abnormalities, or disorientation, comprising administering to a patient in need thereof an effective amount of a disclosed compound.

In some embodiments, the improvement in cognitive or neurological function is measured as a score increase between about 1% and 20% in the delayed verbal recall task of the revised Wechsler Memory Scale. For example, the improvement in cognitive function may be measured as a score increase between about 10% and 20%, or between about 10% and 15%, or between about 5% and 15%.

In some embodiments, the present invention provides a method of treating an alcohol-related disorder such as ethanol toxicity, hangover, and neurological and other effects of excessive alcohol consumption. In some embodiments, the alcohol-related disorder is selected from Wemicke-Korsakoff Syndrome, alcoholic neuropathy, alcohol withdrawal syndrome, alcoholic cerebellar degeneration, and alcoholic myopathy. In a large epidemiological survey of headache in Danish 25- to 64-year-olds, the lifetime prevalence of hangover headache was 72 percent, making it the most common type of headache reported. Alcohol intoxication results in vasodilatation, which may induce headaches. Alcohol has effects on several neurotransmitters and hormones that are implicated in the pathogenesis of headaches, including histamine, serotonin, and prostaglandins. The alcohol withdrawal syndrome following the cessation of excessive drinking results from compensatory changes in the central nervous system that take place in response to chronically administered depressant substances (in this case, alcohol, or more specifically, ethanol). These changes include alterations in two types of receptors embedded in nerve cell membranes. One receptor type binds with an important chemical messenger (i.e., neurotransmitter) called gamma-aminobutyric acid (GABA), and the other type binds with another neurotransmitter, glutamate. Both GABA and glutamate are critical in regulating nerve cell activity: GABA is the body's primary means of inhibiting nerve cell activity, and glutamate is the primary means of exciting it. Following chronic alcohol exposure, the body decreases (i.e., downregulates) the number or sensitivity of GABA receptors and increases (i.e., upregulates) the number or sensitivity of glutamate receptors in an effort to counterbalance alcohol's sedative effects. When alcohol is removed from the body, however, the central nervous system and the portion of the nervous system that coordinates response to stress (i.e., the sympathetic nervous system) remain in an unbalanced “overdrive” state.

### Addictive Disorders

Disclosed compounds are also useful in treating addictions, addictive behaviors, behavioral addictions, compulsive disorders and behaviors, and related conditions.

The use of certain compounds in treating such addictions, behaviors, and disorders is described in WO/2019/157317, the contents of which are hereby incorporated by reference.

Cocaine self-administering mice exhibit significantly higher glutamate levels in the VTA (ventral tegmental area) of the brain. The VTA, in particular the VTA dopamine neurons, serve several functions in the reward system, motivation, cognition, and drug addiction, and may be the focus of several psychiatric disorders. The elevated glutamate levels appear to be due, at least in part, to loss of glutamate uptake into astrocytes. Without wishing to be bound by theory, it is believed that reduced availability of glutamate has negative effects on astrocyte function and this loss of function affects neuronal activity and drug-seeking behavior. It has now been found that the compounds disclosed herein treat or prevent relapse in addicted individuals, for example by reversing such loss of astrocyte function. Such loss of astrocyte function may be partly due to reduced expression of the glutamate transporter (GLT-1) in astrocytes. Since astrocytes metabolize glutamate to produce ATP, this likely impairs glutamate uptake, weakens astrocyte oxidative metabolism and downstream ATP-dependent processes and thereby weakens their ability to maintain an optimal environment for VTA neuronal activity.

Accordingly, in one aspect, the present invention provides a method of preventing, ameliorating, treating, or promoting recovery from an addiction, addictive behavior, behavioral addiction, brain reward system disorder, compulsive disorder, or related condition, comprising administering to a subject in need thereof an effective amount of a compound described herein, or a pharmaceutically acceptable salt thereof or composition comprising the same.

In some embodiments, the addiction is to an addictive substance. In some embodiments, the addictive substance is a prescription or recreational drug.

In some embodiments, the addictive substance is selected from alcohol, nicotine, a stimulant, a cannabinoid agonist, or an opioid agonist. In some embodiments, the addictive substance is selected from heroin, cocaine, alcohol, an inhalant, an opioid, nicotine, an amphetamine, or a synthetic analog, salt, composition, or combination thereof.

In some embodiments, the amphetamine is selected from bupropion, cathinone, MDMA, or methamphetamine.

In some embodiments, the prescription or recreational drug is selected from a cannabinoid agonist or opioid agonist.

In some embodiments, the addiction is an alcohol or nicotine addiction.

In some embodiments, the subject is a polydrug abuser.

In some embodiments, the prescription or recreational drug is selected from cocaine, heroin, bupropion, cathinone, MDMA, or methamphetamine morphine, oxycodone, hydromorphone, fentanyl, or a combination thereof.

In some embodiments, a disclosed compound increases energy metabolism mediated by astrocytes, such as astrocyte mitochondria. In some embodiments, the compound reverses loss of glutamate uptake into astrocytes caused by a substance with abuse potential. In some embodiments, the compound at least partially reverses the remodeling of the brain reward system caused by the addiction. In some embodiments, such effects are mediated by brain or CNS adenosine A₃ receptors, such as astrocyte A₃R in the VTA; or microglia A₃R.

In another aspect, the present invention provides a method of preventing, ameliorating, treating, or promoting recovery from an addiction, addictive behavior, behavioral addiction brain reward system disorder, compulsive disorder, or related condition by increasing energy metabolism mediated by astrocytes, glia, microglia, neurons, endothelium cells, or other cells of the brain and/or CNS, comprising administering to a subject in need thereof an effective amount of a compound described herein, or a pharmaceutically acceptable salt thereof or composition comprising the same.

In some embodiments, the method treats or prevents a relapse of an addiction or addictive behavior in the subject. In some embodiments, the subject is addicted to one or more addictive substances such as addictive drugs (drugs having abuse potential). As described below, such drugs include prescription drugs and recreational drugs such as heroin, cocaine, nicotine, or an opioid agonist.

In another aspect, the present invention provides a method of treating or preventing withdrawal caused by addiction to one or more addictive substances or drugs, comprising administering to a subject in need thereof an effective amount of a compound described herein, or a pharmaceutically acceptable salt thereof or composition comprising the same. In some embodiments, the compound decreases withdrawal symptoms in an addicted individual in withdrawal. In some embodiments, the compound treats withdrawal in an addicted individual in withdrawal. In some embodiments, the method further comprises co-administering another drug for treating withdrawal and, optionally, counseling such as psychotherapy. In some embodiments, the method further comprises a cognitive behavioral therapy. In some embodiments, the method further comprises a digital therapeutic. Digital therapeutics include, for example, reSET or reSET-O (Pear Therapeutics).

In some embodiments, the present invention provides a method of treating or preventing a relapse of a compulsive disorder or compulsive behavior, comprising administering to a subject in need thereof an effective amount of a compound described herein, or a pharmaceutically acceptable salt thereof or composition comprising the same.

In some embodiments, the compulsive disorder is obsessive-compulsive disorder (OCD). Tourette syndrome, trichotillomania, anorexia, bulimia, anxiety disorder, psychosis, or post-traumatic stress disorder.

According to another aspect, the present invention provides a method for treating one or more behavioral addictions and addictive behaviors or disorders comprising administering to a subject in need thereof a compound described herein, or a pharmaceutically acceptable salt thereof or composition comprising the same. Behavioral addictions and addictive disorders result from the intoxication one senses from the release of brain chemicals (e.g., serotonin, adrenaline, epinephrine, etc.) during certain activities. Such disorders are known in the art and include gambling, sex addiction, pornography addiction, eating disorders, spending addiction, rage/anger, workaholism, exercise addiction, risk taking addictions (e.g. kleptomania and pyromania), perfectionism, internet or video game addiction, and compulsive use of electronic devices such as texting and checking social media, to name a few.

As used herein, the term “addiction” includes, unless otherwise specified, physical or psychological dependence on a substance. Addiction may involve withdrawal symptoms or mental or physical distress if the substance is withdrawn. Addiction includes drug liking, drug dependence, habit-formation, neurological and/or synaptic changes, development of brain reward system disorders, behavioral changes, or other signs or symptoms of addiction in a subject.

As used herein, the term “addictive drug” or “drug having abuse potential” includes drugs and other substances such as nicotine, whether approved by a regulatory body for treatment of a disease or not, that are known to result in clinical, behavioral, or neurological manifestations of addiction or compulsive behavior. In some embodiments, the addictive drug includes nicotine, a cannabinoid agonist, a stimulant, or an opioid agonist. “Addictive substance” refers to addictive drugs as well as other substances of abuse such as alcohol. Examples of addictive substances thus include heroin, cocaine, alcohol, opiates, nicotine, inhalants, amphetamines, and their synthetic analogs.

### Pain Conditions and Disorders

Disclosed compounds are also useful in treating pain, pain disorders, and related conditions. Accordingly, in one aspect, the present invention provides a method of treating, preventing, promoting recovery from, or ameliorating a pain condition or disorder, comprising administering to a subject in need thereof an effective amount of a compound described herein, or a pharmaceutically acceptable salt thereof or pharmaceutical composition thereof.

In some embodiments, the pain condition or disorder is pain control (pain management, e.g., management of chronic pain). For the use of certain nucleoside and nucleotide compounds in treating this and related conditions, see, for example, US 2010/0256086, hereby incorporated by reference.

In other embodiments, the pain condition or disorder is selected from pain mediated by the CNS, such as neuropathic pain, inflammatory pain, or acute pain. For the use of certain nucleoside and nucleotide compounds in treating these conditions, see, for example, Br J Pharmacol. 2010 March; 159(5):1106-17. Doi: 10.1111/j.1476-5381.2009.00596.x. Epub 2010 Feb. 5. “A comparative analysis of the activity of ligands acting at P2X and P2Y receptor subtypes in models of neuropathic, acute and inflammatory pain.” Andó RD1, Méhész B, Gyires K, Illes P, Sperlágh B. PMID: 20136836, hereby incorporated by reference.

In some embodiments, the pain condition or disorder is migraine.

In some embodiments, the pain condition or disorder is neuropathic pain, inflammatory pain, or acute pain. See, e.g., Tosh, D. K.: Padia, J.; Salvemini, D.; Jacobson, K. A. Efficient, large-scale synthesis and preclinical studies of MRS5698, a highly selective A₃ adenosine receptor agonist that protects against chronic neuropathic pain. Purinergic Signalling 2015, 11, 371-387.

In some embodiments, the pain condition or disorder is central pain syndrome, peripheral neuropathy, corneal neuropathic pain, post stroke pain, or pain caused by multiple sclerosis.

In another aspect, the present invention provides a method of treating pain, comprising administering to a subject in need thereof an effective amount of a disclosed compound, or a pharmaceutically acceptable salt thereof or a pharmaceutically acceptable composition thereof.

In some embodiments, the pain is neuropathic pain. In some embodiments, the pain is inflammatory pain. In some embodiments, the pain is acute pain. In some embodiments, the pain is chronic pain. In some embodiments, the pain is nociceptive pain. In some embodiments, the pain is non-inflammatory musculoskeletal pain, fibromyalgia syndrome (FMS), or myofascial pain syndrome (MPS).

In some embodiments, the pain is selected from musculoskeletal pain, fibromyalgia, myofascial pain, pain during menstruation, pain during osteoarthritis, pain during rheumatoid arthritis, pain during gastrointestinal inflammation, pain during inflammation of the heart muscle, pain during multiple sclerosis, pain during neuritis, pain during AIDS, pain during chemotherapy, tumor pain, headache, chronic pain syndrome (CPS), central pain, trigeminal neuralgia, shingles, stamp pain, phantom limb pain, temporomandibular joint disorder, nerve injury, migraine, post-herpetic neuralgia, neuropathic pain encountered as a consequence of injuries, amputation infections, metabolic disorders or degenerative diseases of the nervous system, neuropathic pain associated with diabetes, pseudesthesia, hypothyroidism, uremia, vitamin deficiencies or alcoholism, acute pain after injuries, postoperative pain, pain during acute gout, and pain from an operation.

In some embodiments, the musculoskeletal pain is neck and shoulder pain and/or spasms, back pain, sciatica, chest ache, or thigh muscle ache.

In some embodiments, the pain is, or is associated with, otitis externa (OE), otitis media (OM), mastoiditis, bullous myringitis, eustachian tubal catarrh, labyrinthitis, facial nerve neuritis, temporal bone osteoradionecrosis, mal de debarquement, temporal bone fracture, or temporomandibular joint disease.

### Pharmaceutically Acceptable Compositions

The basic features of the disclosed pharmaceutical compositions are described above. Additional components may be present in addition to those described above. For example, disclosed compositions may further comprise a pharmaceutically acceptable carrier, adjuvant, or vehicle.

The term “subject” or “patient,” as used herein, means an animal, preferably a mammal, and most preferably a human.

The term “pharmaceutically acceptable carrier, adjuvant, or vehicle” refers to anon-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compounds of the invention are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

In certain embodiments, the compounds of the invention are administered orally at dosage levels of about 0.01 mg/kg to about 50 mg/kg and preferably from about 0.01 mg/kg to about 25 mg/kg, of subject body weight per day, one or more times a day, to obtain the desired therapeutic effect. In certain embodiments, the compounds of the invention are administered orally or parenterally at dosage levels of about 0.01 mg/kg to about 50 mg/kg, or about 0.01 mg/kg to about 25 mg/kg, or about 0.05 mg/kg to about 10 mg/kg, or about 0.05 mg/kg to about 5 mg/kg, or about 0.1 mg/kg to about 2.5 mg/kg, of subject body weight per day, one or more times a day, to obtain the desired therapeutic effect.

In certain embodiments, the compounds of the invention are administered orally at dosage levels of about 0.01 mg/kg to about 50 mg/kg and preferably from about 0.01 mg/kg to about 25 mg/kg, of subject body weight, once an hour every hour, to obtain the desired therapeutic effect. In certain embodiments, the compounds of the invention are administered orally at dosage levels of about 0.01 mg/kg to about 50 mg/kg and preferably from about 0.01 mg/kg to about 25 mg/kg, of subject body weight, once an hour every hour, for 1 to 8 hours, to obtain the desired therapeutic effect. In certain embodiments, the compounds of the invention are administered orally at dosage levels of about 0.01 mg/kg to about 50 mg/kg and preferably from about 0.01 mg/kg to about 25 mg/kg, of subject body weight, once an hour every hour, for 1 to 6 hours, to obtain the desired therapeutic effect. In certain embodiments, the compounds of the invention are administered orally at dosage levels of about 0.01 mg/kg to about 50 mg/kg and preferably from about 0.01 mg/kg to about 25 mg/kg, of subject body weight, once an hour every hour, for 1 to 4 hours, to obtain the desired therapeutic effect. In certain embodiments, the compounds of the invention are administered orally at dosage levels of about 0.01 mg/kg to about 50 mg/kg and preferably from about 0.01 mg/kg to about 25 mg/kg, of subject body weight, once an hour every hour, for 1 to 2 hours, to obtain the desired therapeutic effect.

In certain embodiments, the compounds of the invention are administered orally or parenterally at dosage levels of about 0.01 mg/kg to about 50 mg/kg, or about 0.01 mg/kg to about 25 mg/kg, or about 0.05 mg/kg to about 10 mg/kg, or about 0.05 mg/kg to about 5 mg/kg, or about 0.1 mg/kg to about 2.5 mg/kg, of subject body weight, one or more times a day, to obtain the desired therapeutic effect. In certain embodiments, the compounds of the invention are administered orally or parenterally at dosage levels of about 0.01 mg/kg to about 50 mg/kg, or about 0.01 mg/kg to about 25 mg/kg, or about 0.05 mg/kg to about 10 mg/kg, or about 0.05 mg/kg to about 5 mg/kg, or about 0.1 mg/kg to about 2.5 mg/kg, of subject body weight, once an hour every hour, to obtain the desired therapeutic effect. In certain embodiments, the compounds of the invention are administered orally or parenterally at dosage levels of about 0.01 mg/kg to about 50 mg/kg, or about 0.01 mg/kg to about 25 mg/kg, or about 0.05 mg/kg to about 10 mg/kg, or about 0.05 mg/kg to about 5 mg/kg, or about 0.1 mg/kg to about 2.5 mg/kg, of subject body weight, once an hour every hour, for 1 to 8 hours to obtain the desired therapeutic effect.

In some embodiments, the compound or composition is administered once an hour, every hour. In some embodiments, the compound or composition is administered once an hour, every hour, for 1 to 8 hours. In some embodiments, the compound or composition is administered once, twice, three times, four times, five times, six times, seven times, or eight times over an eight-hour period. In some embodiments, the compound or composition is administered at least once and up to twice, up to three times, up to four times, up to five times, up to six times, up to seven times, or up to eight times over an eight-hour period.

In some embodiments, the compound or composition is administered once an hour or once every two hours.

In some embodiments, the compound or composition is administered twice in a one-hour period followed by less frequent administration. In some embodiments, the compound or composition is administered twice in a one-hour period followed by administration once an hour for up to six additional hours.

In some embodiments, the compound or composition is administered up to three times over five hours. In some embodiments, the compound or composition is administered twice over five hours. In some embodiments, the compound or composition is administered three times over five hours.

In some embodiments, the compound or composition is administered once followed by administration every two hours. In some embodiments, administration is continued indefinitely until a sufficient therapeutic effect is achieved in the subject. In some embodiments, administration is continued for up to two days (including, e.g., about 48 hours).

In addition to the active compounds, the dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol. 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

The pharmaceutical composition may also contain one or more coloring agents. Exemplary coloring agents include: FD&C Blue Nos. 1 and 2, FD&C Green No. 3, FD&C Red Nos. 3 and 40, FD&C Yellow Nos. 5 and 6, Orange B, Citrus Red No. 2, annatto extract, beta-carotene, grape skin extract, cochineal extract or carmine, paprika oleoresin, caramel color, fruit and vegetable juices, saffron, Monosodium glutamate (MSG), hydrolyzed soy protein, autolyzed yeast extract, disodium guanylate or inosinate.

The consumable food additive of the invention may also contain one or more surfactants, such as glycerol monostearate and polysorbate 80.

The pharmaceutical composition may also contain one or more preservatives. Exemplary preservatives include ascorbic acid, citric acid, sodium benzoate, calcium propionate, sodium erythorbate, sodium nitrite, calcium sorbate, potassium sorbate, BHA, BHT, EDTA, and tocopherols.

The pharmaceutical composition may also contain one or more nutrient supplements, such as: thiamine hydrochloride, riboflavin, niacin, niacinamide, folate or folic acid, beta carotene, potassium iodide, iron or ferrous sulfate, alpha tocopherols, ascorbic acid, Vitamin D, amino acids, multi-vitamin, fish oil, co-enzyme Q-10, and calcium.

In some embodiments, the sweetening agent is selected from sucrose, glucose, sucralose, fructose, lactose, aspartame, invert sugar, corn syrup, stevia extract powder, stevioside, steviol, saccharin, saccharin salts, potassium acetosulfam, sorbitol, xylitol, mannitol, erythritol, lactitol, alitame, miraculin, monellin, and thaumatin or a combination of the same. In some embodiments, the flavoring agent is selected from coconut, orange, cherry, fruit punch, lemon, lime, grapefruit, raspberry, tutti-frutti, black cherry, strawberry, mint, peppermint such as Peppermint Naefco, and combinations thereof.

Further exemplary sweetening agents include: sucrose (sugar), glucose, fructose, sorbitol, mannitol, corn syrup, high fructose corn syrup, saccharin, aspartame, sucralose, acesulfame potassium (acesulfame-K), and neotame.

Further flavoring agents include: menthol flavor, eucalyptus, mint flavor and/or L-menthol. Sweetening agents may include one or more of the following: xylitol, sorbitol, isomalt, aspartame, sucralose, acesulfame potassium, and saccharin.

Combinations with Other Therapeutic Agents

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

In certain embodiments, a provided compound, or composition thereof, is administered in combination with other therapeutic agents, such as tissue plasminogen activators and other thrombolytics, blood thinners, statins, ACE inhibitors, angiotensin II receptor blockers (ARBs), beta blockers, calcium channel blockers or diuretics, to a patient in need thereof.

In certain embodiments, the tissue plasminogen activator used in combination with compounds or compositions of the invention include, but are not limited to, alteplase, desmoteplase, reteplase, streptokinase, tenecteplase, urokinase, or combinations of any of the above.

In certain embodiments, the blood thinners used in combination with compounds or compositions of the invention include, but are not limited to, warfarin, heparin, apixabam, clopidogrel, aspirin, rivaroxaban, dabigatran, or combinations of any of the above.

In certain embodiments, the statins used in combination with compounds or compositions of the invention include, but are not limited to, atorvastatin, rosuvastatin, fluvastatin, lovastatin, pravastatin, simvastatin and pitavastatin, cerivastatin, mevastatin, or combinations of any of the above.

In certain embodiments, the ACE inhibitors used in combination with compounds or compositions of the invention include, but are not limited to, captopril, enalapril, fosinopril, lisinopril, moexipril, perindopril, quinapril, ramipril, trandolapril benazepril, or combinations of any of the above.

In certain embodiments, the angiotensin II receptor blockers (ARBs) used in combination with compounds or compositions of the invention include, but are not limited to, azilsartan, candesartan, eprosartan, irbesartan, losartan, olmesartan, telmisartan, valsartan, fimasartan, or combinations of any of the above.

In certain embodiments, the beta blockers used in combination with compounds or compositions of the invention include, but are not limited to, atenolol, bisoprolol, betaxolol, carteolol, carvedilol, labetalol, metoprolol, nadolol, nebivolol, oxprenolol, penbutolol, pindolol, propranolol, timolol, or combinations of any of the above.

In certain embodiments, the calcium channel blockers used in combination with compounds or compositions of the invention include, but are not limited to, dihydropyridines: amlodipine, cilnidipine, clevidipine, felodipine, isradipine, lercanidipine, levamlodipine, nicardipine, nifedipine, nimodipine, nisoldipine, nitrendipine, diltiazem, verapamil, or combinations of any of the above.

In certain embodiments, the diuretics used in combination with compounds or compositions of the invention include, but are not limited to, loop diuretics, thiazide diuretics, thiazide-like diuretics and potassium-sparing diuretics, or combinations of any of the above.

In certain embodiments, the loop diuretics used in combination with compounds or compositions of the invention include, but are not limited to, bumetanide, ethacrynic acid, furosemide, torsemide, or combinations of any of the above.

In certain embodiments, the thiazide diuretics used in combination with compounds or compositions of the invention include, but are not limited to, epitizide, hydrochlorothiazide and chlorothiazide, bendroflumethiazide, methyclothiazide, polythiazide, or combinations of any of the above.

In certain embodiments, the thiazide-like diuretics used in combination with compounds or compositions of the invention include, but are not limited to, indapamide, chlorthalidone, metolazone, or combinations of any of the above.

In certain embodiments, the potassium-sparing diuretics used in combination with compounds or compositions of the invention include, but are not limited to, amiloride, triamterene, spironolactone, eplerenone, or combinations of any of the above.

In certain embodiments, a provided compound, or composition thereof, is administered in combination with a second agent selected from nonsteroidal anti-inflammatory drugs (NSAIDs), corticosteroids, muscle relaxants, anti-anxiety drugs, serotonin receptor agonists, antidepressants, anticonvulsants, and corticosteroids. In some embodiments, the second agent is selected from opioids, triptans, and COX-2 inhibitors. In some embodiments, the second agent is ibuprofen, acetaminophen, aspirin, naproxen, diclofenac, diflunisal, etodolac, fenoprofen, flurbiprofen, indomethacin, ketorolac, mefenamic acid, meloxicam, nabumetone, oxaprozin, piroxicam, sulindac, tolmetin, celecoxib, a triptan, oxycodone, morphine, codeine, hydromorphone, oxymorphone, fentanyl, or sufentanil. In some embodiments, the second agent is acetaminophen with codeine (Tylenol #2, #3, #4), buprenorphine, fentanyl transdermal patches, hydrocodone with acetaminophen, hydrocodone with ibuprofen, hydrocodone, hydromorphone, meperidine, methadone, morphine, morphine sustained-release (e.g., MS-Contin®, Avinza®, Kadian®) oxycodone sustained-release (e.g., OxyContin®), Oxycodone with acetaminophen (e.g., Percocet®) oxycodone with aspirin (e.g., Percodan®), oxycodone with ibuprofen (e.g., Combunox®), oxymorphone (e.g., Opana®, Opana ER®), pentazocine, propoxyphene with aspirin, propoxyphene with acetaminophen, tapentadol (e.g., Nucynta®), Nucynta ER®)), tramadol, tramadol with acetaminophen (Ultram, Ultracet), a mixed opioid agonist/antagonist such as pentazocine/naloxone (e.g., Talwin NX®) butorphanol, or nalbuphine (e.g., Nubain®), an antidepressant such as amitriptyline (e.g., Elavil®), bupropion, desipramine (e.g., Norpramin®) duloxetine (e.g., Cymbalta®), imipramine (e.g., Tofranil®), venlafaxine (e.g., Effexor®), an anticonvulsant such as carbamazepine (e.g., Tegretol®), clonazepam (e.g., Klonopin®), gabapentin (e.g., Neurontin®), lamotrigine (e.g., Lamictal), pregabalin (e.g., Lyrica®), tiagabine (e.g., Gabitril®), or topiramate (e.g., Topamax®)), a fibromyalgia medication such as milnacipran (e.g., Savella®), an anxiolytic such as alprazolam (e.g., Xanax®), diazepam (e.g., Valium®), lorazepam (e.g., Ativan®), triazolam (e.g., Halcion®), a muscle relaxant such as baclofen (e.g., Lioresal®), carisoprodol (e.g., Soma®), chlorzoxazone (e.g., Parafon Forte®, DSC), cyclobenzaprine (e.g., Flexeril®), dantrolene (e.g., Dantrium®), metaxalone (e.g., Skelaxin®) methocarbamol (e.g., Robaxin®), orphenadrine (e.g., Norflex®), or tizanidine (e.g., Zanaflex®), a corticosteroid such as cortisone, prednisone, prednisolone, dexamethasone, methylprednisolone (e.g., Medrol®, A-Methapred®, Depo Medrol®, Solu Medrol®), or triamcinolone. In some embodiments, the second agent is a triptan (serotonin receptor agonist) or other migraine treatment, such as almotriptan (e.g., Axert®), eletriptan (e.g., Relpax®), frovatriptan (e.g., Frova®), naratriptan ((e.g., Amerge®), rizatriptan (e.g., Maxalt®), sumatriptan (e.g., Imitrex®), sumatriptan/naproxen sodium (e.g., Treximet®), zolmitriptan (e.g., Zomig), erenumab (e.g., Aimovig®), or fremanezumab (e.g., Ajovy®), meclizine, dimenhydrinate, ubrogepant (e.g., Ubrelvy®)), or botulinum toxin type A (e.g., Botox®®)).

In certain embodiments, a provided compound, or composition thereof, is administered in combination with a mechanical thrombectomy device, to a patient in need thereof. In certain embodiments, the mechanical thrombectomy device is a stroke thrombectomy device or a coil embolization device for cerebral aneurysm. In certain embodiments, such a device includes, but is not limited to, a coil retriever, an aspiration device or a stent retriever.

In certain embodiments, a combination of 2 or more therapeutic agents may be administered together with compounds or compositions of the invention. In certain embodiments, a combination of 3 or more therapeutic agents may be administered together with compounds or compositions of the invention.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another, normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the present invention, an additional therapeutic agent, and a pharmaceutically acceptable carrier, adjuvant, or vehicle.

The amount of both, a provided compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between about 0.001-100 mg/kg body weight/day of the additional therapeutic agent can be administered, or about 0.001 mg/kg to about 500 μg/kg, or about 0.005 mg/kg to about 250 μg/kg, or about 0.01 mg/kg to about 100 μg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In one embodiment, the present invention provides a composition comprising a compound of the present invention and one or more additional therapeutic agents. The therapeutic agent may be administered together with a compound of the present invention, or may be administered prior to or following administration of a compound of the present invention. Suitable therapeutic agents are described in further detail below. In certain embodiments, a compound of the present invention may be administered up to 5 minutes. 10 minutes, 15 minutes, 30 minutes, 1 hour, 2 hours, 3 hours, 4 hours, 5, hours, 6 hours, 7 hours, 8 hours, 9 hours, 10 hours. 11 hours. 12 hours, 13 hours, 14 hours, 15 hours, 16 hours, 17 hours, or 18 hours before the therapeutic agent. In other embodiments, a compound of the present invention may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours. 15 hours. 16 hours, 17 hours, or 18 hours following the therapeutic agent.

In some embodiments, the present invention provides a medicament comprising at least one compound of the present invention or a pharmaceutically acceptable salt thereof and a pharmaceutically acceptable carrier.

All features of each of the aspects of the invention apply to all other aspects mutatis mutandis.

In order that the invention described herein may be more fully understood, the following examples are set forth. It should be understood that these examples are for illustrative purposes only and are not to be construed as limiting this invention in any manner.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Example 1—General Preparation of Compound A

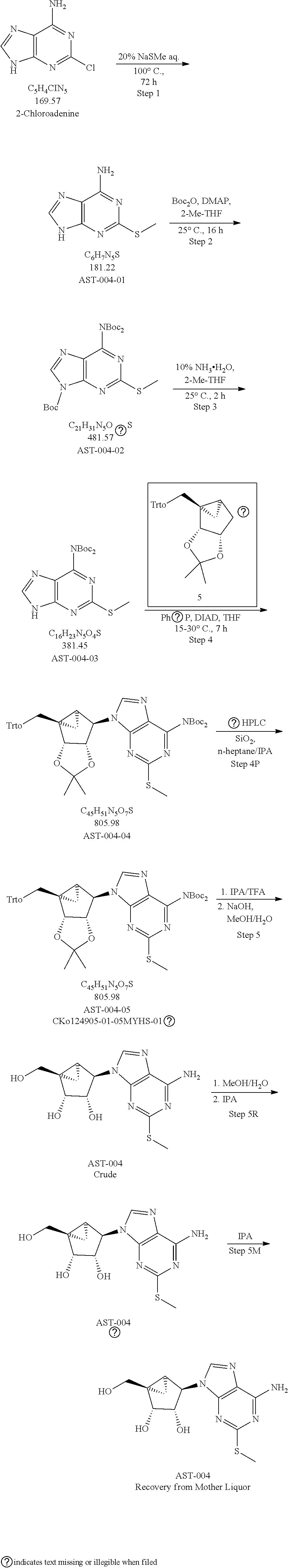

The formation of the methylthioether (AST-004-01) used 6 equivalents of sodium thiol methane (NaSMe) under aqueous conditions, at 90-105° C. for approximately 72 hours. Upon reaction completion by IPC, the reaction mixture was cooled to 15-30° C. and the pH was adjusted to 8-9 with hydrochloric acid. The mixture was heated to 70-80° C. for 30-60 minutes and then cooled to 15-25° C. for 2-3 hours. The mixture was filtered, washed with acetonitrile, then dried.

### Steps 2 and 3

Prior to the reaction, the Step 1 product (AST-004-01) was azeotropically dried to remove residual water in 2-methyl tetrahydrofuran (2-MeTHF). The formation of the tri-Boc protected intermediate (AST-004-02) used 5 equivalents of di-tert-butyl dicarbonate (Boc₂O) and 0.3 equivalents of N,N-dimethylpyridin-4-amine (DMAP) at 25° C. for 16 hours in 2-methyl tetrahydrofuran (2-MeTHF) to form the tri-Boc protected intermediate (AST-004-02) in situ. Upon reaction completion by IPC, the reaction mixture was cooled to 0-10 C, water was added, the mixture was filtered, and the organic layer was separated, washed with water and separated again. The Boc group in the 9 position was removed by reacting with 10% ammonium hydroxide at 0-5 C to form the di-Boc protected intermediate, AST-004-03. Upon reaction completion by IPC, the pH was adjusted to 7-8 hydrochloric acid and the organic portion is separated and then concentrated. N-heptane is added, heated to 60˜70° C. for 30 to 60 minutes, cooled to 15˜25° C. for ˜2 to 3 hours and the product was filtered, washed with n-heptane, then dried. The Step 3 product (AST-004-03) can be recrystallized a second time from 2-MeTHF and n-heptane to increase purity of the intermediate if needed.

### Steps 4 and 4P

Step 4 utilized the Mitsunobu reaction to couple the methanocarba moiety to the di-Boc protected intermediate (AST-004-03) from Step 3. Equal equivalents of di-Boc protected intermediate (AST-004-03) and methanocarba moiety were charged in tetrahydrofuran (THF) to azeotropically dried to remove residual water. Triphenylphosphine (TPP) and diisopropyl azodicarboxylate (DIAD) were added at 1.5 equivalents each at 15-30° C. for approximately 7 hours. A solvent exchange was performed to remove THF and replace it with methyl tert-butyl ether (MTBE). The mixture was then cooled to 0-10° C. for 1 to 2 hours and filtered. The filtrate was washed with MTBE and is collected and concentrated and a solvent exchange was performed to replace MTBE with DCM. The concentrated DCM mixture was used for the Step 4P purification step.

The purification step (Step 4P) used preparative chromatography to purify the Step 4 crude product. Step 4P started by concentrating the crude Step 4 product in dichloromethane. Once concentrated, the solution was diluted with n-heptane for loading on a silica column. Prior to the preparative chromatography, two use-tests were performed to confirm elution time of the desired product (approximately 37 to 55 minutes). Small preparative chromatography batches were prepared and each batch was purified by column chromatography with a SiO₂ sorbant and n-heptane/isopropyl alcohol in a mobile phase gradient over 141 minutes. All fractions of the peak of interest were tested, pooled, concentrated, and solvent exchanged with isopropyl alcohol.

### Step 5 Crude

The purified product was charged and was concentrated under reduced pressure and temperature. An excess of trifluoroacetic acid (TFA) was added for the global deprotection and heated to 60° C. for 17 hours. The reaction was cooled, and MTBE was added. The mixture was further cooled between 0° C. and 10° C. for 2 to 3 hours and the solids were filtered and washed with MTBE. The crude TFA salt was stirred for 1 to 2 hours at 15° C.-30° C. and was cooled between 0° C. and 10° C. for 2 to 3 hours and the solids filtered and washed with MTBE for a second time. Methanol and water for injection (WFI) were added and the pH is adjusted to pH 8-9 with aqueous sodium hydroxide. The solvent was concentrated and water for injection was added. This solvent mixture was concentrated until methanol is less than 3%. The mixture was heated to 80-90° C. for 1 to 2 hours then cooled to 10 to 20° C. and crystalized for 1 to 3 hours. The solids were filtered, and the filter cake washed with WFI and dried with nitrogen.

### Step 5R Recrystallization of AST-004 Drug Substance

The crude API was dissolved in 16 volumes of 50/50 methanol and WFI and heated to 50° C. to 60° C. for 1 to 2 hours. The mixture was cooled to 20° C. to 40° C. and passed through a bed of diatomaceous earth and a capsule filter. The solvent mixture was concentrated under reduced pressure and temperature ≤60° C. The solvent was exchanged with isopropanol until water was less than 3%. The mixture was cooled to 0° C. to 10° C. for 2 to 3 hours to crystalize the AST-004. The solids were filtered and the filter cake was washed with isopropanol. The AST-004 solids were dried on the filter for 31 hours with nitrogen. The material was sieved through 20 mesh screen and packaged.

Step 5M Recovery of API from Mother Liquor

Additional API may be recovered from the mother liquor following a similar process as Step 5 Recrystallization. The mother liquor from the Step 5 Recrystallization was exchanged with isopropanol until water content was ≤3% and concentrated to 5 to 10 solvent volumes under reduced pressure and temperature ≤60° C. The mixture was cooled to 0° C. to 5° C. for ˜13 hours to crystalize additional AST-004. The AST-004 solids were filtered and the cake was washed with isopropanol. The solids were dried on the filter for 24 hours with nitrogen. The material was sieved through 20 mesh screen and packaged.

Powder x-ray powder diffraction (XRPD) shows a crystalline material of Form B. Two crystal forms (Form A and Form B) have been observed. Form B was selected for the clinical development of AST-004. All released lots of AST-004 have been Form B. Form A is a hemihydrate. It was used in in early development and exploratory toxicology. The manufacturing process as described yields only Form B, the anhydrous form. A bridging study was conducted and concluded that Form B was the most stable form when water activity is ≤63% and Form A is the most stable form when the water activity is ≥72%.

### Example 2—Preparation of Free Base Form A and Form B of Compound A

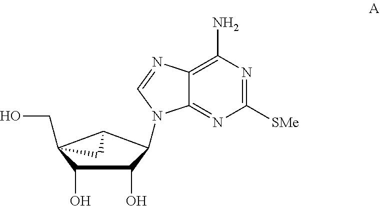

Compound A exists in two polymorphs, as described above. Either polymorph may be used in the pharmaceutical compositions described herein. Form A and Form B may be prepared using methods described in U.S. Pat. No. 10,765,693, which is hereby incorporated by reference. Such methods include those reproduced below.

### Form A of Compound A—Preparation Method 1

Compound 13 of Scheme 3 of U.S. Pat. No. 10,765,693 (350 g, 0.58 mol), i.e., the acetonide produced from Step 4P in the scheme above, and water (2 L) was added to a 3 L round bottom flask. 200 mL of trifluoroacetic acid (TFA) was added and the mixture was stirred at 60° C. for 12 hrs. HPLC showed the reaction was complete. The mixture was cooled to room temperature and washed with by methylene chloride (MC, 300 mL*3) and concentrated. The basic resin Amberlyst 21 was added to the mixture to achieve a pH of about 9 and the mixtures was stirred for 16 hours at room temperature. The mixture was then filtered, washed with methanol (MeOH) and concentrated. Purification by column chromatography was carried out [SiO₂(5×), MC/MeOH=15/1 to 5/1 (100 V)) to yield Compound A as light yellow solid. Compound A was then dissolved in HPLC-grade MeOH to a clear solution and rotary evaporated (water pump, bath temperature 35° C.). Form A of compound A was formed gradually during the concentration. After most of the MeOH was removed (no weight change), deionized water was added and rotary evaporated to dryness three times (oil pump, bath temperature 35° C.) to remove residual MeOH. The obtained compound was further dried by rotary evaporation (oil pump, bath temperature 35° C.) until no weight change (˜16 hours), yielding Form A of compound A.

### Form A of Compound A—Preparation Method 2

To a plastic grinding cup was added 18.4 mg of Compound A and 10 μL of water. A stainless-steel ball was added. The sample was then milled on a Retsch mill at 100% power for 20 minutes at room temperature.

### Form A of Compound A—Preparation Method 3

To a 20 mL glass vial was added 200.0 mg of Compound A and 3.0 mL of water. A magnetic stir bar was added and the vial was capped. The vial was placed on a heating/stirring plate and the slurry was stirred magnetically for 7 days at ambient temperature. The vial was centrifuged for 10 minutes and the mother liquor was decanted. A dry air purge was directed into the vial for 10 minutes to dry the solid. The vial was then placed in a room temperature vacuum desiccator for 2 hours.

### Form A of Compound A—Preparation Method 4

To a 20 mL glass vial was added 200.0 mg of Compound A and 3.0 mL of water. A magnetic stir bar was added and the vial was capped. The vial was placed on a heating/stirring plate set at 60° C. and the slurry was stirred magnetically for 3 days. The vial was centrifuged for 10 minutes and the mother liquor was decanted. A dry air purge was directed into the vial for 10 minutes to dry the solid. The vial was then placed in a room temperature vacuum desiccator for 2 hours.

Table 1, supra, is reproduced below and sets forth the X-ray diffraction peaks observed for Form A of compound A.

TABLE 1
XRPD Peak Positions for Form A of Compound A.

| | Relative |
|---|---|
| °2θ | Intensity |
| 7.6 | 1.49 |
| 8.0 | 100 |
| 9.0 | 3.49 |
| 10.8 | 4.60 |
| 11.8 | 1.59 |
| 12.5 | 4.59 |
| 13.1 | 59.02 |
| 16.2 | 34.56 |
| 16.7 | 37.68 |
| 17.2 | 10.65 |
| 17.9 | 45.59 |
| 18.1 | 16.05 |
| 18.3 | 10.91 |
| 19.8 | 4.96 |
| 21.0 | 30.62 |
| 21.5 | 35.63 |
| 22.8 | 2.30 |
| 23.1 | 6.16 |
| 23.7 | 4.21 |
| 23.9 | 9.39 |
| 24.9 | 54.39 |
| 26.1 | 32.89 |
| 26.5 | 13.26 |
| 26.6 | 22.22 |
| 27.1 | 60.62 |
| 28.6 | 10.52 |
| 29.3 | 1.66 |
| 29.7 | 9.05 |
| 30.1 | 1.89 |
| 30.4 | 1.77 |
| 30.8 | 12.79 |
| 32.0 | 8.32 |
| 32.8 | 8.27 |
| 32.9 | 13.11 |
| 33.7 | 9.73 |
| 34.7 | 4.31 |
| 36.3 | 3.98 |
| 36.7 | 8.82 |
| 37.9 | 12.68 |
| 38.2 | 3.16 |
| 38.5 | 1.75 |
| 38.7 | 2.83 |
| 39.6 | 2.37 |
| — | — |
| — | — |

### Form B of Compound A—Preparation Method 1

To a 20 mL glass vial was added 200.4 mg of Form A of Compound A and 3.0 mL of acetone. A magnetic stir bar was added and the vial was capped. The vial was placed on a heating/stirring plate set at 60° C. and the slurry was stirred magnetically for 3 days. The vial was centrifuged for 10 minutes and the mother liquor was decanted. A dry air purge was directed into the vial for 10 minutes to dry the solid. The vial was then placed in a room temperature vacuum desiccator for 2 hours.

### Form B of Compound A—Preparation Method 2

To a 1-dram glass vial was added 17.2 mg of Form A of Compound A and 1 mL of ethyl acetate. The slurry was stirred magnetically on a heating/stirring plate set to 60° C. and ethanol (absolute) was added until dissolution occurred (added 1 mL). The stir bar was removed, the vial capped, and the heat turned off. Once cooled to ambient temperature, no solid was observed and the vial was left at ambient temperature overnight, during which time crystallization did not occur. The vial was then placed in a refrigerator at about 5° C. and left for 3 days, during which time crystallization did not occur. The vial was then placed in a freezer at about −15° C. and left for 2 days, during which time crystallization occurred. The solid was recovered by filtration.

### Form B of Compound A—Preparation Method 3

To a plastic grinding cup was added 18.4 mg of Form A of Compound A and 10 μL of ethanol. A stainless-steel ball was added. The sample was then milled on a Retsch mill at 100% power for 20 minutes.

### Form B of Compound A—Preparation Method 4

To a 1-gram glass vial was added 3.8 mg of Form A of Compound A and 0.7 mL of tetrahydrofuran. The solid dissolved. The vial was then placed into a 20 mL glass vial containing 2 mL of hexanes. The 20 mL vial was capped and the sample left at ambient temperature for 6 days, during which crystallization occurred. The solvent was decanted and the solids allowed to air dry.

Table 2, supra, is reproduced below and sets forth the X-ray diffraction peaks observed for Form B of compound A.

TABLE 2
XRPD Peak Positions for Form B of Compound A.

| | Relative |
|---|---|
| °2θ | Intensity |
| 4.7 | 2.43 |
| 7.6 | 3.96 |
| 9.5 | 14.73 |
| 10.0 | 10.55 |
| 10.5 | 7.20 |
| 13.8 | 20.73 |
| 14.2 | 10.53 |
| 14.7 | 40.39 |
| 15.2 | 6.59 |
| 15.4 | 14.70 |
| 16.2 | 4.80 |
| 17.1 | 24.58 |
| 17.9 | 58.03 |
| 18.3 | 12.94 |
| 19.0 | 100 |
| 20.1 | 88.35 |
| 21.0 | 42.70 |
| 21.5 | 68.69 |
| 23.0 | 2.13 |
| 23.8 | 53.95 |
| 24.3 | 8.09 |
| 24.6 | 3.80 |
| 25.4 | 5.74 |
| 25.6 | 8.51 |
| 25.9 | 35.31 |
| 26.2 | 20.76 |
| 26.6 | 16.53 |
| 27.6 | 7.29 |
| 28.8 | 25.11 |
| 29.1 | 8.40 |
| 29.5 | 4.27 |
| 29.9 | 3.19 |
| 30.2 | 6.20 |
| 30.6 | 7.95 |
| 31.6 | 4.20 |
| 32.3 | 1.42 |
| 32.7 | 5.03 |
| 33.1 | 4.67 |
| 33.6 | 3.56 |
| 35.9 | 7.11 |
| 37.0 | 2.87 |
| 37.4 | 1.78 |
| 39.0 | 1.26 |
| — | — |
| — | — |

### Example 3—Preparation of an Oral Disintegrating Tablet of Compound A

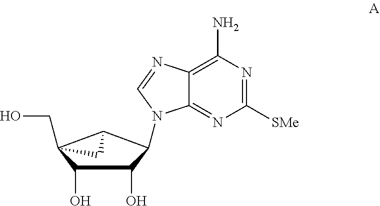

### Example 3A: First Study

In a first attempt to develop an oral disintegrating table comprising Compound A, two batches of tablets at dose strengths of 50 mg and 150 mg in 300 mg and 900 mg wet-fill weights respectively were prepared and the incorporation of Compound A into a gelatin matrix was evaluated. The in-process stability and finished product properties of the tablets were also assessed.

Product was dosed into product packaging wells on completion of mixing (0 hours of solution hold; SH0) and after 24 hours of solution hold (SH24) to assess the stability of the batches over the hold period.

Critical physical attributes of the dried units were evaluated (e.g., appearance and dispersion time). Samples were also tested to gather indicative in-process drug substance stability information.

Approximately 20 g of unmicronised Compound A was divided into two batches to be manufactured, one to be assessed in a bovine gelatin matrix at room temperature and the other to be assessed in fish HMW gelatin at ˜12 degrees Celsius.

The feasibility studies were carried out using Compound A. Study 1 (max. 5 batches) focused on the following aims:
Microscopy on raw material and zero-hour solution hold (0SH) mix, 0SH mix (post-homogenisation if required) and at 24SH.
Identify the preliminary API concentration and unit size, appropriate for a 50 mg and 150 mg dose strength
Assess API incorporation into the gelatin mix
Identify additional formulation components as required based on available technical data and customer requirements
Identify product specific processing variables for evaluation based on available technical data (e.g., suspension hold time, freezing temperature)
Assess units for physical attributes (appearance and dispersion time) and chemical attributes (assay, related substances)

The formulations evaluated are summarised in Table 3.

TABLE 3
Formulations Evaluated

| Batch Details | Formulation A-1 | Formulation A-2 |
|---|---|---|
| Purified Water (w/w %) | 75.53 | 77.23 |
| Compound A (w/w %) | 16.67 | 16.67 |
| Gel NonOx EP/USNF/JP | — | 3.30 |
| (Gelita) (w/w %) | | |
| Mannitol (w/w %) | 3.30 | 2.50 |
| Sucralose (w/w %) | 0.10 | 0.10 |
| Flavour Peppermint Naefco | 0.20 | 0.20 |
| 957.685 (w/w %) | | |
| Gelatin EP/USP/JP | 4.20 | — |
| (Fish HMW) (w/w %) | | |

Drug Product materials: Purified water was purified using a Purelab Option Elga DV25 purification device. Gel NonOx EP/USNF/JP (Gelita) was obtained from commercial sources. Mannitol was obtained from commercial sources. Sucralose was obtained from commercial sources. Gelatin EP/USP/JP (Fish HMW) was obtained from commercial sources.

Tablet blister pack materials: The pack-forming material was a 165 mm 5-layer AAB film. The sealing material was a sachet of 110 mm×170 mm.

Formulations A-1 and A-2 were made by making a pre-mix of gelatin and water. The resultant pre-mix was heated to 60±2° C. for a minimum of 10 minutes and then cooled down to 23±2° C. The sucralose and peppermint were then added and fully dissolved. All excipients incorporated easily. Compound A was then wetted as the premix was added dropwise, and the pH was measured. The resulting suspensions were held at temperature and dosing at 23° C.±2° C. (GelNonOx) or 12° C.±2° C. (HMW fish gelatin). Batch A-1 was transferred to a water bath set at 12±2° C. with stirring. Batch A-2 was transferred to a hotplate stirrer and held at ambient temperature with stirring. After approximately half an hour, the opaque white mixes had visible agglomerates and settling around the circumference of the vessel, with the whole mix appearing more viscous. Formulations were unsuitable for dosing without additional manual mixing with a spatula. After 24 hours of stirring, the opaque white mix, still had visible agglomerates and settling around the circumference of the vessel. Formulations were again unsuitable for dosing without additional manual mixing. Formulations were unsuitable for dosing without additional manual mixing with a spatula, upon manual mixing, questions were raised regarding uniformity of the mix, however, dosing was still commenced. All batches had no other problems reported during dosing. The mixes were dosed at two different dosing points: i) Initial timepoint, after the completion of the mix; solution hold 0 (SH0) and ii) after 24 hours of stirring; solution hold 24 hours (SH24). After the initial dosing time point, batch 1 was left stirring in the water bath set at 12±2° C. and batch 2 was left stirring on a hotplate stirrer held at ambient temperature.

After the hold time, each resulting suspension was dispensed by an Eppendorf dosing pump into the wells of blister packs with a 300 mg or a 1200 mg pocket size, where each well was filled with 300 mg±2% (294 mg to 306 mg) or wells of blister packs with a 300 mg or a 1200 mg pocket size, where each well was filled with 1200 mg±2% (882 mg to 918 mg), respectively. The filled wells were frozen in a freeze tunnel at −80° C. for 3 minutes and 15 seconds, then stored frozen at −15° C. for 12 hours minimum. The filled frozen wells were subjected to lyophilization at 0° C. for 12 hours. The filled lyophilized wells were evaluated while in dry storage, and then were sealed for sacheting with an HSE 3 Heat Sealer at 160° C. with a pressure of 50-54 psi and a 0.3 second dwell time. No issues were reported during sacheting.

Table 4 summarizes the dosing points, the number of blisters dosed and the frozen hold per batch.

TABLE 4
Mix Preparation for Batches A-1 and A-2

| | Dosing | Frozen | Number of |
|---|---|---|---|
| Batch ref | time point | hold time* | Blisters Dosed |
| A-I-a | SH0 | FH 12 hr 01 min | 1 |
| (Fish gelatin, | | | |
| 50 mg dose) | | | |
| A-I-b | | FH 12 hr 01 min | 1 |
| (Fish gelatin, | | | |
| 150 mg dose) | | | |
| A-2-a | | FH 12 hr 01 min | 1 |
| (Bovine gelatin, | | | |
| 50 mg dose) | | | |
| A-2-b | | FH 12 hr 01 min | 1 |
| (Bovine gelatin, | | | |
| 150 mg dose) | | | |
| A-I-a | SH24 | FH 12 hr 34 min | 1 |
| (Fish gelatin, | | | |
| 50 mg dose) | | | |
| A-I-b | | FH 12 hr 34 min | 1 |
| (Fish gelatin, | | | |
| 150 mg dose) | | | |
| A-2-a | | FH 12 hr 01 min | 1 |
| (Bovine gelatin, | | | |
| 50 mg dose) | | | |
| A-2-b | | FH 12 hr 01 min | 1 |
| (Bovine gelatin, | | | |
| 150 mg dose) | | | |

*FH—Frozen hold

TABLE 5
pH Data for Batches B-100-1 and B-100-2

| Batch | pH at end of mixing | pH of Mix at SH24 |
|---|---|---|
| A-1 | 9.80 | 8.60 |
| A-2 | 9.14 | 8.45 |

No significant pH differences were observed during mix hold, indicating good stability over the 24 hours solution hold time.

TABLE 6
Macroscopic Observations for batches A-1 and A-2

| Batch | Appearance at SH0 | Appearance at SH24 |
|---|---|---|
| A-1 | Opaque white mix with | Opaque white mix with |
| | visible particles, settling | visible particles, settling |
| | observed around the | observed around the |
| | circumference of the mixing | circumference of the mixing |
| | vessel. | vessel. |
| A-2 | Opaque white mix with | Opaque white mix with |
| | visible particles, settling | visible particles, settling |
| | observed around the | observed around the |
| | circumference of the mixing | circumference of the mixing |
| | vessel. | vessel. |

No significant differences in macroscopic appearance were noted between batches formulated with fish gelatin when compared to those formulated with bovine gelatin.

TABLE 7
Finished Product Observations for Batches A-1 and A-2

| | Appearance of Dried Product | |
|---|---|---|
| Batch | SH0 | SH24 |
| A-1-a | Good, white units with an | Good, white units with an |
| 0SH | uneven matte top surface | uneven matte top surface |
| (Fish gelatin, | and matte base. Units are | and matte base. Units are |
| 50 mg dose) | easily removed from pockets | easily removed from pockets |
| | with no residue remaining. | with no residue remaining. |
| | Good deboss. No defects | Good deboss. No defects |
| | reported. | reported. |
| A-1-b | Good, white units with an | Good, white units with an |
| 0SH | uneven matte top surface and | uneven matte top surface and |
| (Fish gelatin, | matte base. Units are easily | matte base. Units are easily |
| 150 mg dose) | removed from pockets with | removed from pockets with |
| | no residue remaining. Good | no residue remaining. Good |
| | deboss. 2/6 Cracks. | deboss. No defects reported. |
| A-2-a | Good, white units with an | Good, white units with an |
| 0SH | uneven matte top surface | uneven matte top surface |
| (Bovine gelatin, | and matte base. Units are | and matte base. Units are |
| 50 mg dose) | easily removed from pockets | easily removed from pockets |
| | with no residue remaining. | with no residue remaining. |
| | Good deboss. No defects | Good deboss. |
| | reported. | 1/30 Cracks. |
| A-2-b | Good, white units with an | Good, white units with an |
| 0SH | uneven matte top surface and | uneven matte top surface and |
| (Bovine gelatin, | matte base. One unit broke | matte base. Units are easily |
| 150 mg dose) | on removal, leaving very | removed from pockets with |
| | minor residue in pocket. | no residue remaining. Good |
| | Good deboss. | deboss. |
| | 6/6 Cracks. | 3/6 Cracks. |

Finished product appearance for all batches and solution hold times was good, with minimum number of defects. Some sporadic major or minor craked units were observed. No drying defects such as melting were observed, this is also an indication that both the frozen hold and the drying conditions are suitable for this stage of development.

TABLE 8
Dispersion Time Data for Batches A-1 and A-2

| | | Mean | Range |
|---|---|---|---|
| | | Dispersion | Dispersion |
| | | Time | Time |
| Batch | Sub-batch | (seconds) | (seconds) |
| B-100-1a | SH0 | 22.99 | 16.91-29.06 |
| (Fish gelatin, | SH24 | 1.86 | 1.00-2.72 |
| 50 mg dose) | | | |
| B-100-1b | SH0 | 79.39 | 75.21-83.56 |
| (Fish gelatin, | SH24 | 2.34 | 2.25-2.43 |
| 150 mg dose) | | | |
| B-100-2a | SH0 | 2.39 | 2.06-2.72 |
| (Bovine gelatin, | SH24 | 1.52 | 1.46-1.57 |
| 50 mg dose) | | | |
| B-100-2b | SH0 | 2.06 | 1.82-2.30 |
| (Bovine gelatin | SH24 | 2.22 | 2.10-2.34 |
| 150 mg) | | | |

The dispersion time is an in-process test. The units were placed bottom surface facing down in a beaker filled with purified water at 20±5° C. The length of time for the unit to fully wet was timed using a calibrated stopwatch. This process was carried out for five units in total.

Most of the batches dispersed within acceptable limits of ≤10 seconds. Batch 1 at SH0 had dispersion times outside of the acceptable limits of ≤10 seconds. No significant differences in dispersion times were noted between units with differing formulations and dose strengths with the SH24 batches.

### Conclusions for Study 1:

Formulations A-1 and A-2 initially produced smooth opaque mixes. However, after approximately half an hour, the opaque white mix had visible agglomerates and settling around the circumference of the vessel, with the whole mix appearing more viscous. This may be due to Compound A undergoing a change in its crystal form, from an anhydrous form to a hemi-hydrate form.

Formulations were dosed successfully. However, content uniformity may be impacted by viscous/agglomerated appearance of mix post solid-state transition that occurred shortly after mixing. Different formulations, Bovine versus Fish gelatin (or possibly the associated mix temperature), appear to change the rate of crystal state transitions.

No evident or significant macroscopic or viscosity changes were observed over the solution holding period. However, it is probable that Compound A has transitioned to a different crystal form soon after it was added to the mix and prior to the initial samples being taken for microscopy.

Finished product appearance was good for all batches with minimum defects both for initial timepoint and for the 24 hours solution hold samples. Defects such as cracking are are to be resolved at a subsequent stage of development

Most batches dispersed within acceptable limits of ≤10 seconds. No significant differences in dispersion times were noted between units with differing formulations, dose strengths or solution hold times.

Unit reconstitution study confirmed that freeze-drying has not had any significant effects on the particle size and morphology.

Dosage delivery results were over the target weight and quite variable, which was expected to be due to the viscous and partly agglomerated mix following the Compound A crystal change. There are no observable related substances which indicates good in-process stability.

### Example 3B: Second Study

Study 2 was conducted with the aim of focusing on the following factors:
Assessment of the effect of gelatin type, API micronization, mix homogenization and mix temperature (10° C., ambient, 40° C.) on the particle size of the recrystallised API in suspension.
Observation of the morphology and particle size of the raw API (micronized and non-micronized AST-004) under the microscope.
Dosing selective batches based on having the smallest API particle size, good visual mix homogeneity and acceptable mix appearance (e.g., a suspension mix with no visible agglomeration). Units were to be dosed at 0-hour and 24-hour Suspension Hold (SH) timepoints for the micronized API batches, and the 0-hour SH timepoint only for the non-micronized API batches. In-process testing (pH, particle size analysis, macro- and microscopic observations) was to be conducted at approximately similar timepoints.
Assessment of two wet fill weights (400 mg and 1200 mg) corresponding to 50 mg and 150 mg dose strengths of micronized AST-004 in the final units.
Assessment of one wet fill weight (400 mg) corresponding to the 50 mg dose strength of non-micronized Compound A in the final units.
Observation of the appearance and measurement of the dispersion times of the finished product.
Assay and related substances testing on selective samples of finished product to be conducted.

The formulation details for this study are summarized in Table 9. Materials and methods are the same as in Example 3A.

TABLE 9
Formulation Details.

| | Formulation Details (% w/w) | | | | | |
|---|---|---|---|---|---|---|
| | B-129-1 | | B-129-2 | | B-130-1* | B-130-2* |
| Dose | 50 mg | 150 mg | 50 mg | 150 mg | 50 mg | 50 mg |
| Strength | | | | | | |
| Unit Size | 400 mg of | 1200 mg of | 400 mg of | 1200 mg of | 400 mg of | 400 mg of |
| | suspension | suspension | suspension | suspension | suspension | suspension |
| | dosed in | dosed in | dosed in | dosed in | dosed in | dosed in |
| | 500 mg | 1200 mg | 500 mg | 1200 mg | 500 mg | 500 mg |
| | pocket | pocket | pocket | pocket | pocket | pocket |
| Micronized | 12.50 | | | | — | |
| AST-004 | | | | | | |
| Non- | — | | | | 12.50 | |
| micronized | | | | | | |
| AST-004 | | | | | | |
| HMW Fish | 4.20 | | — | | 4.20 | — |
| Gelatin | | | | | | |
| NonOx | | | 3.30 | | — | 3.30 |
| Bovine | | | | | | |
| Gelatin | | | | | | |
| Mannitol | 3.30 | | 2.50 | | 3.30 | 2.50 |
| Sucralose | 0.10 | | | | | |
| Peppermint | 0.20 | | | | | |
| Flavor | | | | | | |
| Purified | 79.70 | | 81.40 | | 79.70 | 81.40 |
| Water** | | | | | | |

*Formulations for the non-micronized API batches were determined based on the outcomes from the micronized API sub-batches.
**Purified water is removed during freeze drying.

Micronized Compound A was used in batches B-129-1 to B-129-10, while non-micronized Compound A was used in batches B-130-1 and B-130-2. The formulations described above were used to make the following batches:

TABLE 10
Formulation Details for Batches B-129/1-10 (Micronized
API) and B-130/1-2 (Non-Micronized API)

| | Batch Number | |
|---|---|---|
| | B-129-1b | |
| | Batch Conditions | |
| | Fish Gelatin, 10° C., Homogenized | |
| Material | % w/w | Weight Dispensed (g) |
| Purified Water | 79.70* | 131.25 g aliquot of |
| Gelatin USP/EP/JP (Fish | 4.20 | batch B-129-1 |
| HMW) | | |
| Mannitol EP/USP | 3.30 | |
| Sucralose Micronized | 0.10 | |
| USNF/EP (ALAB) | | |
| Flavor Peppermint Naefco | 0.20 | |
| 957.685 | | |
| Micronized AST-004 | 12.50 | 18.75 |
| Total | 100.00 | 150.00 |

| | Batch Number | |
|---|---|---|
| | B-129-2b | |
| | Batch Conditions | |
| | Bovine Gelatin, Ambient, Homogenized | |
| Material | % w/w | Weight Dispensed (g) |
| Purified Water | 81.40* | 131.25 g aliquot of |
| Gel NonOx EP/USNF/JP | 3.30 | batch B-129-2 |
| (Gelita) | | |
| Mannitol EP/USP | 2.50 | |
| Sucralose Micronized | 0.10 | |
| USNF/EP (ALAB) | | |
| Flavor Peppermint Naefco | 0.20 | |
| 957.685 | | |
| Micronized AST-004 | 12.50 | 18.75 |
| Total | 100.00 | 150.00 |
| | Batch Number | |
| | B-129-3 | |
| | Batch Conditions | |
| | Fish Gelatin, Ambient, Homogenized | |
| Material | % w/w | Weight Dispensed (g) |
| Purified Water | 79.70* | 131.25 g aliquot of |
| Gelatin USP/EP/JP (Fish | 4.20 | batch B-129-1 |
| HMW) | | |
| Mannitol EP/USP | 3.30 | |
| Sucralose Micronized | 0.10 | |
| USNF/EP (ALAB) | | |
| Flavour Peppermint Naefco | 0.20 | |
| 957.685 | | |
| Micronized AST-004 | 12.50 | 18.75 |
| Total | 100.00 | 150.00 |

| | Batch Number | |
|---|---|---|
| | B-129-4 | |
| | Batch Conditions | |
| | Bovine Gelatin, Ambient, Stirred | |
| Material | % w/w | Weight Dispensed (g) |
| Purified Water | 81.40* | 131.25 g aliquot of |
| Gel NonOx EP/USNF/JP | 3.30 | batch B-129-1 |
| (Gelita) | | |
| Mannitol EP/USP | 2.50 | |
| Sucralose Micronized | 0.10 | |
| USNF/EP (ALAB) | | |
| Flavour Peppermint Naefco | 0.20 | |
| 957.685 | | |
| Micronized AST-004 | 12.50 | 18.75 |
| Total | 100.00 | 150.00 |

| | Batch Number | |
|---|---|---|
| | B-129-5 | |
| | Batch Conditions | |
| | Fish Gelatin, 40° C., Stirred | |
| Material | % w/w | Weight Dispensed (g) |
| Purified Water | 79.70* | 131.25 g aliquot of |
| Gelatin USP/EP/JP (Fish | 4.20 | batch B-129-1 |
| HMW) | | |
| Mannitol EP/USP | 3.30 | |
| Sucralose Micronized | 0.10 | |
| USNF/EP (ALAB) | | |
| Flavour Peppermint Naefco | 0.20 | |
| 957.685 | | |
| Micronized AST-004 | 12.50 | 18.75 |
| Total | 100.00 | 150.00 |
| | Batch Number | |
| | B-129-6 | |
| | Batch Conditions | |
| | Fish Gelatin, 10° C., Stirred | |
| Material | % w/w | Weight Dispensed (g) |
| Purified Water | 79.70* | 131.25 g aliquot of |
| Gelatin USP/EP/JP (Fish | 4.20 | batch B-129-1 |
| HMW) | | |
| Mannitol EP/USP | 3.30 | |
| Sucralose Micronized | 0.10 | |
| USNF/EP (ALAB) | | |
| Flavour Peppermint Naefco | 0.20 | |
| 957.685 | | |
| Micronized AST-004 | 12.50 | 18.75 |
| Total | 100.00 | 150.00 |
| | Batch Number | |
| | B-129-7 | |
| | Batch Conditions | |
| | Bovine Gelatin, 40° C., Stirred | |
| Material | % w/w | Weight Dispensed (g) |
| Purified Water | 81.40* | 131.25 g aliquot of |
| Gel NonOx EP/USNF/JP | 3.30 | batch B-129-2 |
| (Gelita) | | |
| Mannitol EP/USP | 2.50 | |
| Sucralose Micronized | 0.10 | |
| USNF/EP (ALAB) | | |
| Flavour Peppermint Naefco | 0.20 | |
| 957.685 | | |
| Micronized AST-004 | 12.50 | 18.75 |
| Total | 100.00 | 150.00 |

| | Batch Number | |
|---|---|---|
| | B-129-8 | |
| | Batch Conditions | |
| | Bovine Gelatin, 40° C., Homogenized | |
| Material | % w/w | Weight Dispensed (g) |
| Purified Water | 81.40* | 131.25 g aliquot of |
| Gel NonOx EP/USNF/JP | 3.30 | batch B-129-2 |
| (Gelita) | | |
| Mannitol EP/USP | 2.50 | |
| Sucralose Micronized | 0.10 | |
| USNF/EP (ALAB) | | |
| Flavour Peppermint Naefco | 0.20 | |
| 957.685 | | |
| Micronized AST-004 | 12.50 | 18.75 |
| Total | 100.00 | 150.00 |

| | Batch Number | |
|---|---|---|
| | B-129-9 | |
| | Batch Conditions | |
| | Fish Gelatin, Ambient, Stirred | |
| Material | % w/w | Weight Dispensed (g) |
| Purified Water | 79.70* | 131.25 g aliquot of |
| Gelatin USP/EP/JP (Fish | 4.20 | batch B-129-2 |
| HMW) | | |
| Mannitol EP/USP | 3.30 | |
| Sucralose Micronized | 0.10 | |
| USNF/EP (ALAB) | | |
| Flavor Peppermint Naefco | 0.20 | |
| 957.685 | | |
| Micronized AST-004 | 12.50 | 18.75 |
| Total | 100.00 | 150.00 |

| | Batch Number | |
|---|---|---|
| | B-129-10 | |
| | Batch Conditions | |
| | Fish Gelatin, 40° C., Homogenized | |
| Material | % w/w | Weight Dispensed (g) |
| Purified Water | 79.70* | 131.25 g aliquot of |
| Gelatin USP/EP/JP (Fish | 4.20 | batch B-129-1 |
| HMW) | | |
| Mannitol EP/USP | 3.30 | |
| Sucralose Micronized | 0.10 | |
| USNF/EP (ALAB) | | |
| Flavor Peppermint Naefco | 0.20 | |
| 957.685 | | |
| Micronized AST-004 | 12.50 | 18.75 |
| Total | 100.00 | 150.00 |
| | Batch Number | |
| | B-130-1 | |
| | Batch Conditions | |
| | Fish Gelatin, 40° C., Homogenized | |
| Material | % w/w | Weight Dispensed (g) |
| Purified Water | 79.70* | 131.25 g aliquot of |
| Gelatin USP/EP/JP (Fish | 4.20 | batch B-129-1 |
| HMW) | | |
| Mannitol EP/USP | 3.30 | |
| Sucralose Micronized | 0.10 | |
| USNF/EP (ALAB) | | |
| Flavor Peppermint Naefco | 0.20 | |
| 957.685 | | |
| Non-micronized AST-004 | 12.50 | 18.75 |
| Total | 100.00 | 150.00 |

Batch Number
B-130-2
Batch Conditions
Bovine Gelatin Pre-mix, 40° C., Homogenized

| Material | % w/w | Weight Dispensed (g) |
|---|---|---|
| Purified Water | 81.40* | 131.25 g aliquot of |
| Gel NonOx EP/USNF/JP | 3.30 | batch B-129-2 |
| (Gelita) | | |
| Mannitol EP/USP | 2.50 | |
| Sucralose Micronized | 0.10 | |
| USNF/EP (ALAB) | | |
| Flavor Peppermint Naefco | 0.20 | |
| 957.685 | | |
| Non-micronized AST-004 | 12.50 | 18.75 |
| Total | 100.00 | 150.00 |

*Purified water is removed during freeze drying.

The following process was used to dispense and mix batches B-129-1 to B-129-10:
All excipients for the stock pre-mixes were dispensed (batches B-129-1 and B-129-1-2).
Gelatin and mannitol were added to the purified water in a stainless steel vessel (SSV) with stirring.
The solution was heated to 60° C.±2° C., and held for 10 minutes with stirring.
The pre-mix of batch B-129-1 was cooled to 10° C.±2° C. in the water bath and batch B-129-2 was cooled to ambient temperature using the hotplate stirrer.
Add the sucralose and peppermint to the pre-mixes.
The pre-mix of batch B-129-1 was dispensed into six aliquots of 131.25 g in individual SSVs. These sub-batches were labeled B-129-1b, B-129-3, B-129-5, B-129-6, B-129-9 and B-129-10. Sub-batches B-129-1b and B-129-6 were placed in a water bath set at 10° C.±2° C. with stirring.
The pre-mix of batch B-129-2 was dispensed into four aliquots of 131.25 g in individual SSVs. Label these sub-batches B-129-2b, B-129-4, B-129-7 and B-129-8.
Sub-batches B-129-2b, B-129-3, B-129-4 and B-129-9 were placed on a hotplate stirrer at ambient temperature (note, temperature monitored but not controlled for mixes held at ambient).
Sub-batches B-129-5, B-129-7, B-129-8 and B-129-10 were placed on a hotplate stirrer at 40° C.±2° C.
Once the pre-mix was at the required temperature, the micronized Compound A was added to the vortex of the pre-mix inside the fume hood and as quickly as practicable. Sub-batches B-129-4, B-129-5, B-129-6, B-129-7 and B-129-9, were stirred using magnetic follower with the same RPM and follower size for each batch.
Sub-batches B-129-1b. B-129-2b, B-129-3, B-129-8 and B-129-10 were homogenized for 15 minutes at 7100 rpm.
All sub-batches were maintained at the required mix temperature (10° C., ambient or 40° C.) with stirring
The morphology and particle size of the raw micronized Compound A was assessed under the microscope.
Samples of mix from each sub-batch for microscopy and particle size analysis were taken at the 0-hour SH. The pH and macroscopic appearance of each mix were recorded. These steps were repeated for the 24-hour SH timepoint.

The following process was used to dispense and mix batches B-130-1 and B-130-2:
131.25 g of the pre-mix of batch B-129-1 was dispensed into an SSV.
131.25 g of the pre-mix from batch B-129-2 was dispensed into an SSV.
The pre-mix aliquots were labelled to indicate whether they were batch B-130-1 or B-130-2, respectively.
Each pre-mix aliquot was heated to 40° C.±2° C. on a hotplate stirrer.
The non-micronized Compound A was added to the vortex of each pre-mix as quickly as practicable.
A sample of mix from each batch was taken after approximately the first 10 seconds following Compound A addition. The morphology and particle size of the Compound A in these samples was assessed under the microscope.
The mixes were homogenized for 15 minutes at 7100 rpm.
The two batches were maintained at the required mix temperature of 40° C.±2° C. with stirring.
The morphology and particle size of the raw non-micronized Compound A was assessed under the microscope.
Samples of mix from each sub-batch for microscopy and particle size analysis were taken at the 0-hour SH. The pH and macroscopic appearance of each mix were recorded.

### Observations of Micronized API Batches:

Initially the API was added to the pre-mixes of sub-batches B-129-4 (bovine gelatin, ambient, stirring) and B-129-9 (fish gelatin, ambient, stirring) while stirring. The API wetted within 1 minute for both mixes. Subsequently the API was added to the pre-mix of sub-batch B-129-6 (fish gelatin, 10° C., stirring), and fully wetted within 4 minutes. However, following the wetting of API in the first three sub-batches, all mixes began to convert to a semi-solidified state. Although the mixes of sub-batches B-129-4 and B-129-9 could be reverted to a suspension-like state upon agitation with a spatula and adjustment of the stirrer speed, the mix of sub-batch B-129-6 remained in a semi-solidified state. It was expected that this was caused by the lower mix temperature of B-129-6, making the mix more viscous.

The API was added to the remaining sub-batches and fully wetted within 1 minute. In accordance with sub-batch B-129-6, the mix of sub-batch 1b (fish gelatin, 10° C., homogenized) began to solidify following API wetting (prior to homogenisation where applicable) and could not be recovered back to the suspension-like state. In contrast, the mix of sub-batches B-129-5 (fish gelatin, 40° C. stirred), B-129-7 (bovine gelatin, 40° C., stirred). B-129-2b (bovine gelatin, ambient, homogenized), B-129-3 (fish gelatin, ambient, homogenized), B-129-8 (bovine gelatin, 40° C., homogenized) and B-129-10 (fish gelatin, 40° C., homogenized) did not convert to a semi-solidified state after API wetting.

Sub-batches B-129-1b, B-129-2b, B-129-3, B-129-8 and B-129-10 were progressed for homogenization. Each mix was homogenized for 15 minutes at 7100 rpm. Despite homogenizing the mix of batch B-129-1b, this did not improve the mix consistency, which consequently remained viscous.

In a further attempt to reduce the viscosity of sub-batches B-129-1b and B-129-6 and to recover both mixes to a suspension-like state, the temperature of the mixes was increased to ambient temperature. However, no improvement in the mixes was observed. The mixes were therefore not progressed beyond the 0-hour in-process testing.

Following the mixing process, all mixes held at 40° C. (sub-batches B-129-5, B-129-7, B-129-8 and B-129-10) were cooled to ambient prior to dosing.

### Observations of Non-Micronized API Batches:

In light of the observations from the micronized API batches, the formulation and mixing method selected for batches B-130-1 and B-130-2 (non-micronized API batches) was based on sub-batches B-129-10 (fish gelatin, 40° C., homogenized) and B-129-8 (bovine gelatin, 40° C., homogenized), respectively. Similar to the micronized API batches, the non-micronized API was added to the vortex of batch B-130-1 and 2 as quickly as practicable. Following the first 10 seconds after API addition, samples of each mix were additionally taken and assessed under the microscope. Both mixes were observed to fully wet within 1 minute of API addition.

The mixes of sub-batches B-130-1 and B-130-2 were subsequently homogenized for 15 minutes at 7100 rpm. However, in contrast to the corresponding micronized API sub-batches B-129-8 and B-129-10, the mixes began to solidify immediately following homogenization; the viscosity change was more pronounced for sub-batch B-130-2. Both mixes were recovered when the mixes were returned to the hotplate stirrer set at 40° C.±2° C. and a spatula was used to re-agitate the mixes back to suspension-like state.

Both mixes were cooled to ambient temperature prior to dosing the blister pockets.

The following process was used to dose and freeze-dry all sub-batches using the same equipment and materials as described in Example 3A:
Sub-batches were dosed into blister pockets. Dosing time was measured at 0SH and 24SH for micronized API sub-batches and only at 0SH for non-micronized API batches.
The dosed blister pockets were frozen at −80° C. for 03:15 (min:sec).
The frozen product remained in a frozen hold for 12 h prior to freeze drying.
The frozen product was lyophilized at 0° C. for 720 min.
The dried product was inspected and tested for dispersion time testing (2 units per sub-batch).
The blisters were heat sealed in sachets using the HSE-3 heat sealer.

TABLE 11
Finished Product Observations for Micronized and Non-Micronized API Batches

| Batch | Description |
|---|---|
| B-129-2b | The quality of the units was acceptable. Units |
| 0SH | were off-white in color with a matte finish. Units |
| Bovine gelatin ambient homogenized | removed easily from the blister pockets with no |
| Micronized API | residue remaining. Units had a good deboss. |
| 400 mg wet fill weight | Cracks: 56/64 |
| (50 mg dose) | No other defects were reported. |
| B-129-2b | The quality of the units was acceptable. Units |
| 24SH | were off-white in color with a matte finish. Units |
| Bovine gelatin ambient homogenized | removed easily from the blister pockets with no |
| Micronized API | residue remaining. Units had a good deboss. |
| 400 mg wet fill weight | No defects were reported. |
| (50 mg dose) | |
| B-129-3 | The quality of the units was acceptable. Units |
| 0SH | were off-white in color with a matte finish. Units |
| Fish gelatin ambient homogenized | removed easily from the blister pockets with no |
| Micronized API | residue remaining. Units had a good deboss. |
| 400 mg wet fill weight | Cracks: 1/64 |
| (50 mg dose) | Major Nodules: 1/64 |
| | No other defects were reported. |
| B-129-3 | The quality of the units was acceptable. Units |
| 24SH | were off-white in color with a matte finish. Units |
| Fish gelatin ambient, homogenized | removed easily from the blister pockets with no |
| Micronized API | residue remaining. Units had a good deboss. |
| 400 mg wet fill weight | No defects were reported. |
| (50 mg dose) | |
| B-129-4 | The quality of the units was acceptable. Units |
| 0SH | were off-white in color with a matte finish. Units |
| Bovine gelatin ambient | removed easily from the blister pockets with no |
| stirring | residue remaining. Units had a good deboss. |
| Micronized API | No defects were reported. |
| 400 mg wet fill weight | |
| (50 mg dose) | |
| B-129-4 24SH | The quality of the units was acceptable. Units |
| Bovine gelatin ambient | were off-white in color with a matte finish. Units |
| stirring | removed easily from the blister pockets with no |
| Micronized API | residue remaining. Units had a good deboss. |
| 400 mg wet fill weight | No defects were reported. |
| (50 mg dose) | |
| B-129-5 | The quality of the units was acceptable. Units |
| 0SH | were off-white in color with a matte finish. Units |
| Fish gelatin | removed easily from the blister pockets with no |
| 40° C. | residue remaining. Units had a good deboss. |
| stirring | No defects were reported. |
| Micronized API | |
| 400 mg wet fill weight | |
| (50 mg dose) | |
| B-129-5 | The quality of the units was acceptable. Units |
| 24SH | were off-white in color with a matte finish. Units |
| Fish gelatin | removed easily from the blister pockets with no |
| 40° C. | residue remaining. Units had a good deboss. |
| stirring | No defects were reported. |
| Micronized API | |
| 400 mg wet fill weight | |
| (50 mg dose) | |
| B-129-7 | The quality of the units was acceptable. Units |
| 0SH | were off-white in color with a matte finish. Units |
| Bovine gelatin 40° C. | removed easily from the blister pockets with no |
| stirring | residue remaining. Units had a good deboss. |
| Micronized API | No defects were reported. |
| 400 mg wet fill weight | |
| (50 mg dose) | |
| B-129-7 | The quality of the units was acceptable. Units |
| 24SH | were off-white in color with a matte finish. Units |
| Bovine gelatin 40° C. | removed easily from the blister pockets with no |
| stirring | residue remaining. Units had a good deboss. |
| Micronized API | No defects were reported. |
| 400 mg wet fill weight | |
| (50 mg dose) | |
| B-129-8 | The quality of the units was acceptable. Units |
| 0SH | were off-white in color with a matte finish. Units |
| Bovine gelatin 40° C. | removed easily from the blister pockets with no |
| homogenized | residue remaining. Units had a good deboss. |
| Micronized API | Cracks: 5/64 |
| 400 mg wet fill weight | No other defects were reported. |
| (50 mg dose) | |
| B-129-8 | The quality of the units was acceptable. Units |
| 24SH | were off-white in color with a matte finish. Units |
| Bovine gelatin 40° C. | removed easily from the blister pockets with no |
| Homogenized | residue remaining. Units had a good deboss. |
| Micronized API | No defects were reported. |
| 400 mg wet fill weight | |
| (50 mg dose) | |
| B-129-9 | The quality of the units was acceptable. Units |
| 0SH | were off-white in color with a matte finish. Units |
| Fish gelatin ambient | removed easily from the blister pockets with no |
| stirring | residue remaining. Units had a good deboss. |
| Micronized API | Cracks: 8/64 |
| 400 mg wet fill weight | No other defects were reported. |
| (50 mg dose) | |
| B-129-9 | The quality of the units was acceptable. Units |
| 24SH | were off-white in color with a matte finish. Units |
| Fish gelatin ambient | removed easily from the blister pockets with no |
| stirring | residue remaining. Units had a good deboss. |
| Micronized API | No defects were reported. |
| 400 mg wet fill weight | |
| (50 mg dose) | |
| B-129-10 | The quality of the units was acceptable. Units |
| 0SH | were off-white in color with a matte finish. Units |
| Fish gelatin | removed easily from the blister pockets with no |
| 40° C. homogenized | residue remaining. Units had a good deboss. |
| Micronized API | Cracks: 3/64 |
| 400 mg wet fill weight | No other defects were reported. |
| (50 mg dose) | |
| B-129-10 | The quality of the units was acceptable. Units |
| 24SH | were off-white in color with a matte finish. Units |
| Fish gelatin | removed easily from the blister pockets with no |
| 40° C. homogenized | residue remaining. Units had a good deboss. |
| Micronized API | Cracks: 2/64 |
| 400 mg wet fill weight | No other defects were reported. |
| (50 mg dose) | |
| B-130-1 | The quality of the units was acceptable. Units |
| 0SH | were off-white in color with a matte finish. Units |
| Fish gelatin | removed easily from the blister pockets with no |
| 40° C. homogenized | residue remaining. Units had a good deboss. |
| Non-Micronized API | No defects were reported. |
| 400 mg wet fill weight | |
| (50 mg dose) | |
| B-130-2 | The quality of the units was acceptable. Units |
| 0SH | were off-white in color with a matte finish. Units |
| Bovine gelatin 40° C. homogenized | removed easily from the blister pockets with no |
| Non-Micronized API | residue remaining. Units had a good deboss. |
| 400 mg wet fill weight | Cracks: 22/32 |
| (50 mg dose) | No other defects were reported. |

The finished product quality was acceptable across all batches. Units were off-white in color with a matte appearance and were easy to remove from the blister pockets with no residue remaining. The deboss was also good. Some cracks were reported in selective batches; however, this is not a cause for concern at this stage of development as the formulation has not yet been optimized.

Dispersion times were measured as in Example 3A.

TABLE 12
Dispersion Time Data for Micronized
and Non-micronized API batches

| | Suspension | Mean (n = 2) | Range (n = 2) |
|---|---|---|---|
| Sub-batch | Hold (Hours) | (seconds) | (seconds) |
| B-129-2b | 0 | <1 | <1 |
| | 24 | <1 | <1 |
| B-129-3 | 0 | <1 | <1 |
| | 24 | <1 | <1 |
| B-129-4 | 0 | <1 | <1 |
| | 24 | <1 | <1 |
| B-129-5 | 0 | <1 | <1 |
| | 24 | <1 | <1 |
| B-129-7 | 0 | <1 | <1 |
| | 24 | <1 | <1 |
| B-129-8 | 0 | <1 | <1 |
| | 24 | <1 | <1 |
| B-129-9 | 0 | <1 | <1 |
| | 24 | <1 | <1 |
| B-129-10 | 0 | <1 | <1 |
| | 24 | <1 | <1 |
| B-130-1 | 0 | <1 | <1 |
| B-130-2 | 0 | <1 | <1 |

The dissolution times were acceptable. However, the formation of large, needle-shaped particles of the API during formulation represented a significant challenge.

TABLE 13
Summary of Particle Size Information determined from
photomicrographs for micronized API sub-batches

| | Particle size (μm) | |
|---|---|---|
| | 0- | 24- |
| | hour | hour |
| Sub-Batch details | SH | SH |
| B-129-1b | 45-60 | N/A* |
| Fish gelatin, 10° C., homogenized | | |
| B-129-2b | 40-76 | 15-60 |
| Bovine gelatin, ambient, homogenized | | |
| B-129-3 | 60-100 | 20-80 |
| Fish gelatin, ambient, homogenized | | |
| B-129-4 | 60-165 | 16-160 |
| Bovine gelatin, ambient, stirring | | |
| B-129-5 | 65-160 (needle-like), | 16-160 |
| Fish gelatin, 40° C., stirring | 4 (more spherical). | |
| B-129-6 | 50-120 | N/A* |
| Fish gelatin, 10° C., stirring | | |
| B-129-7 | 40-80 (needle-like), 4 | 5-140 |
| Bovine gelatin, 40° C., stirring | (more spherical). | |
| B-129-8 | 45-80 | 15-80 |
| Bovine gelatin, 40° C., homogenized | | |
| B-129-9 | 35-100 (some >120). | 20-120 |
| Fish gelatin, ambient, stirring | | |
| B-129-10 | 70-88 | 30-80 |
| Fish gelatin, 40° C., homogenized | | |

*Due to difficulties in extracting a sample of mix due to its viscous nature, measurements were not taken at the 24-hour SH timepoint.

Based on the results of this study, it can be concluded that a change to the hydrate form of AST-004 still occurred rapidly, regardless of the formulation or mixing processing conditions used or starting API form. For all mixes assessed, the final particle size of the hydrate form to which the API converted to remained approximately constant between the batches. Although some slight trends could be identified between the particle size and processing condition, they do not appear to be significant.

The hydrate particles that formed were also large and acicular; at the 0-hour SH timepoint all mixes contained particles of at least 70 μm in length, with some batches containing particles greater than 100 μm. Large needle-shaped particles are problematic for the manufacturing process because they can increase the risk of particles settling or “webbing” in the dosing lines, resulting in blockages and/or content uniformity issues. Consequently, an upper particle size limit of 25 μm is typically advised for the process. Despite the fact there appeared to be a general reduction in the particle size over the suspension hold period, the particles remained quite large and were mostly above 25 μm.

An assessment of the suitability of the Malvern Mastersizer to size the particles revealed that the current laser diffraction method is not suitable for AST-004. It would therefore necessitate extensive development and optimization in order to accurately determine the particle size of the API. The laser diffraction method remained sub-optimal despite adding a surfactant to the mixes, in an attempt to reduce the extent of agglomeration. The laser diffraction method, based on equivalent particle volumes, also has limitations with respect to the measurement of needle-shaped particles.

Although a change in API form was evident in all batches, the processing condition did have an impact on the ease of the mixing process. For the micronized API sub-batches, the mixes did not permanently semi-solidify when the mix temperature was held at ambient or 40° C. In contrast, a mixing temperature of 10° C. resulted in semi-solidified mixes that could not be reverted to the suspension state. Homogenization was also found to have a protective effect on the mix consistency for the ambient mixes. However, at 40° C., the mixes remained in a suspension state regardless of whether the batches were initially homogenized or stirred only. A comparison to the non-micronized API batches would further suggest that micronization of the API may be additionally necessary to prevent the mixes from thickening excessively over the hold time.

The finished product quality was good across the batches. Some cracking was observed in certain batches; however, this is not a cause for concern at this stage of development when the formulation has not yet been optimized. Moreover, the fast dispersion times would justify increasing the gelatin content in subsequent formulations to reduce the cracking, without having a significant negative impact on unit dispersion rates.

With the exception of sub-batch B-129-2b dosed at the 24-hour SH timepoint, the mean assay values were within the USP label claim limit. An increase in the mean assay values was also observed over the 24-hour SH period for most sub-batches; however, the results were still within the USP label claim limit. Some related substances were evident in all sub-batches analyzed. In total three related compounds at levels greater than LOQ were detected in each sub-batch, with one related compound (RRT 1.53) responsible for approximately 40 to 50% of the total related compounds in each sub-batch. However, levels did not significantly change over the 24-hour mix hold period.

Owing to the larger size of the hydrate particles that form during the formulation process, the development strategy for compound A is considered complex and challenging. These challenges were overcome, as described in Example 3C below.

### Example 3C: Third Study

Compound A was micronized prior to use in these formulations. The micronized Compound A was formulated in batches 1-4. Compound A hemihydrate was formulated in batches 5 and 6, as detailed in Table 14.

TABLE 14
Formulated Batches (% w/w)

| Batch # | Water | Gelatin | Mannitol | Compound A | Sucralose | Peppermint |
|---|---|---|---|---|---|---|
| C-1 | 78.20 | 5.00 | 4.00 | 12.50 | 0.10 | 0.20 |
| C-2^{a} | 77.20 | 5.00 | 4.00 | 12.50 | 0.10 | 0.20 |
| C-3^{b} | bal | 5.00 | 4.00 | 12.50 | 0.10 | 0.20 |
| C-4^{c} | bal | 5.00 | 4.00 | 12.50 | 0.10 | 0.20 |
| C-5 | 77.85 | 5.00 | 4.00 | 12.85 | 0.10 | 0.20 |
| C-6 | 77.85 | 5.00 | 4.00 | 12.85 | 0.10 | 0.20 |

^{a}This batch also contained PEG 400 in 1.00% w/w.
^{b}The pH was adjusted to 3.50 ± 0.50 by addition of citric acid prior to Compound A addition.
^{c}The pH was adjusted to 8.00 ± 0.50 by addition of sodium hydroxide prior to Compound A addition.

The use of the hemi-hydrate form of compound A resulted in less needle-shaped particles after using the Thinky mixer and pestle and mortar. Batch 5 had the best particle shape consistency and were sized at 11-58 μm at 24SH.

### Example 3D: Fourth Study

The objective of this study was to further investigate the production of Compound A Hemi-Hydrate Zydisk units at a range of wet fill weights whilst increasing the concentration of API. Another aim was to repeat two batches from Study 3 to investigate if the pH of the Zydis® premix pH had any impact on particle size when the anhydrous API was added and converted to the Hemi-Hydrate form. Upon completing manufacturing of all 5 batches, each batch containing the Hemi-Hydrate API was to be as to determine which was most appropriate for further manufacturing.

The formulation cycle 4 manufacture was designed to focus on the following factors:
Evaluation of five finished product formulations at 50 mg dose strengths.
Assessment and comparison of the effect of using both micronised anhydrous Compound A or Compound A Hemi-Hydrate).
Observation of the morphology and particle size of all batches prior and after Thinky Mixing.
Evaluation of the feasibility of manufacturing acceptable finished units when increasing the concentration of API in batches Z5642/158/1-3.
Assessment of effect of pH adjustment of pre-mix of batches Z5642/158/4-5 on particle size and morphology following recrystallisation from micronised to hydrate form.
Evaluation of all batches to decide which will be used in an informal Compound A study in Compound A Stability Study PD0341.

TABLE 15
Study 4 Formulation Summary

| | Z5642/ | Z5642/ | Z5642/ | Z5642/ | Z5642/ |
|---|---|---|---|---|---|
| Material | 158/1 | 158/2 | 158/3 | 158/4 | 158/5 |
| Purified | 77.11 | 73.04 | 69.80 | QS | QS |
| Water | | | | | |
| Gelatin | 5.00 | 4.74 | 4.53 | 5.00 | 5.00 |
| USP/ESP/JP | | | | | |
| (Fish HMW) | | | | | |
| Mannitol | 4.00 | 3.79 | 3.62 | 4.00 | 4.00 |
| EP/USP | | | | | |
| Sucralose | 0.10 | 0.10 | 0.10 | 0.10 | 0.10 |
| Micronised | | | | | |
| USNF/EP | | | | | |
| (ALAB) | | | | | |
| Flavour | 0.20 | 0.20 | 0.20 | 0.20 | 0.20 |
| Peppermint | | | | | |
| Naefco | | | | | |
| 957.685 Jpn | | | | | |
| Citric Acid | N/A | N/A | N/A | Qs pH 3.50 ± | N/A |
| Anhydrous | | | | 0.20 | |
| Sodium | N/A | N/A | N/A | N/A | Qs pH 8.00 ± |
| Hydroxide Ph | | | | | 0.20 |
| Eur/NF/JP | | | | | |
| Micronised | N/A | N/A | N/A | 12.50 | 12.50 |
| Compound A | | | | | |
| Hemi- | 13.59 | 18.13 | 21.75 | N/A | N/A |
| Hydrate | | | | | |
| Compound A | | | | | |

In conclusion, all 5 batches were mixed though only batches Z5642/158/1-3 (Hemi-Hydrate API batches) were dosed. Batches Z5642/158/4-5 (micronised anhydrous API) were repeated from Study 3 which evaluated the addition of anhydrous Compound A to premix with altered pH targets. However, due to their viscous nature, they could not be processed further to be Thinky Mixed and therefore, they were not dosed. Batch Z5642/158/2 was chosen to be used in the future manufacture. This was due to the ease of incorporating the API in the pre-mix. This batch also showed good potential to incorporate larger API quantities in smaller unit sizes in order to reach the same dose strength. Therefore, if a larger dose strength is required e.g. 150 mg, it can be accommodated in a 900 mg tablet rather than a 1.2 g tablet for the formulation used in batch Z5642/158/1.

In summary, the results of this feasibility study confirm that a lead formulation for Compound A [50 mg and 150 mg] has been identified. These formulations have adequate physical and chemical stability suitable for progressing to small-scale stability manufacture. The lead formulation at the end of feasibility is described in Table 16; these dose strengths have been developed in a 300 mg and 900 mg wet fill weight (dose proportional).

TABLE 16
Lead Formulation

| | Formulation | | | |
|---|---|---|---|---|
| Product | | | Qty | Qty |
| (Wet Fill | | | (50 mg/ | (150 mg/ |
| Weight) | Material | % w/w | unit) | unit) |
| Compound A | Gelatin | 4.74 | 2.37 | 7.11 |
| (300 mg and | EP/USP/JP | | | |
| 900 mg) | (Fish HMW) | | | |
| | Mannitol | 3.79 | 1.895 | 5.685 |
| | EP/USP | | | |
| | Hemi-hydrate | 18.02 | 9.01 | 27.03 |
| | Compound A* | | | |
| | Sucralose | 0.10 | 0.05 | 0.15 |
| | Micronised NF | | | |
| | (ALAB) | | | |
| | Flavour | 0.20 | 0.10 | 0.30 |
| | Peppermint | | | |
| | Naefco 957.685 | | | |
| | Purified Water¹ | 73.15 | 36.575 | 109.725 |

*The salt correction factor is based on the CoA provided by the API supplier (WuxiAppTec) which determines the amount of API required in the formulation. Table 7's API % w/w amount was determined from the previous study using COA CDo124905-02-WXY-8316-01. The difference between Table 16's API amount and future manufacture API amounts will be negligible.
¹Water removed during freeze drying.

### Example 4—Bioavailability of Compound A

The purpose of this study was to evaluate the bioavailability of Compound A following administration of the ODT formulation. Animals were administered Compound A, as described in the following table.

### Part I—Procedure

Dosing occurred at 50.75 mg/dose. The ODT was placed on the dog's tongue and the mouth held shut for approximately 30 seconds to allow the material to dissolve. In each case, the tablet quickly formed a white paste that was adhered to the tongue and was not swallowed whole.

Cageside Observations, General signs of toxicity including fecal and urine quality, were completed approximately 1 hour post dose on dose days.

### Sample Collection:

Whole blood samples were collected from all animals as noted in the following table for determination of test article exposure.

TABLE 17
Sample Collection Requirements for Pharmacokinetic Analysis

| | Targeted | | |
|---|---|---|---|
| | Sample | Collection | Collection |
| Timepoints | Volume | Site | Device |
| 15, 30 minutes, | 1 mL ± 10% | Cephalic Vein | Tubes containing |
| 1, 2, 4, 8, and | | | K₂EDTA |
| 24 hours postdose. | | | |

### Plasma Analytical Methods:

Method: LC-MS/MS
Column: Restek Raptor ARC-18 2.7 μm (30×2.1 mm)
Mobile Phase A: 0.1% Formic Acid in Water
Mobile Phase B: 0.1% Formic Acid in Acetonitrile
Ionization: (±) ESI with Ion Spray
Analyte: Compound A
MRM: m/z 324.200/182.200 amu
Internal Standard: Labetalol
MRM: m/z 329.200/294.200 amu
Assay Range (LLOQ and ULOQ): 1-5000 ng/mL

### Pharmacokinetic Evaluation:

Pharmacokinetic concentration-time data for Compound A was analyzed using non-compartmental methods (Phoenix® WinNonlin® Version 8.1) with nominal sampling times and dosages. The following parameters were calculated whenever possible and as data allowed: Volume of distribution, Clearance, t_{l/2}, C₀/Cₘₐₓ, Tₘₐₓ, % F. and AUC. Additional parameters may have been calculated as appropriate. Descriptive statistics were generated using Phoenix WinNonlin.

### Part II—Results

Following bioanalysis, pharmacokinetic calculations were conducted. Individual animal and group mean plasma concentration versus time data are shown in Table 18.

TABLE 18
Individual and Mean Compound A Plasma Concentration
(ng/mL) versus Time Data Following an Oral Disintegrating
Tablet Dose of Compound A to Male Beagle Dogs

| | Time (hr) | | | | | | | |
|---|---|---|---|---|---|---|---|---|
| Animal ID | 0.08 | 0.25 | 0.5 | 1 | 2 | 4 | 8 | 24 |
| 1 | — | 352 | 1690 | 2510 | 2070 | 1160 | 413 | 119 |
| 2 | — | 114 | 622 | 1810 | 1530 | 798 | 248 | 28.8 |
| 3 | — | 356 | 2300 | 2080 | 1650 | 964 | 434 | 132 |
| 4 | — | 309 | 1840 | 2370 | 1990 | 1100 | 319 | 48.3 |
| N | NA | 4 | 4 | 4 | 4 | 4 | 4 | 4 |
| Mean | NA | 283 | 1610 | 2190 | 1810 | 1010 | 354 | 82.0 |
| SD | NA | 114 | 710 | 312 | 261 | 161 | 86.3 | 51.1 |
| CV % | NA | 40.5 | 44.0 | 14.2 | 14.4 | 16.0 | 24.4 | 62.3 |

Note:
NA = Not applicable.

Individual animal and group mean pharmacokinetic parameters following a oral tablet dose of Compound A are shown in Table 19.

TABLE 19
Individual and Mean Pharmacokinetic Parameters
Following a Single Oral Disintegrating Tablet
Dose of Compound A to Male Beagle Dogs

| | | | | | | Percent |
|---|---|---|---|---|---|---|
| | Tₘₐₓ | Cₘₐₓ | AUC₀₋₂₄ | AUC_{inf} | t_{1/2} | F |
| Animal ID | (hr) | (ng/mL) | (hr*ng/mL) | (hr*ng/mL) | (hr) | (%)^{b} |
| 1 | 1.0 | 2510 | 14300 | 15300 | 5.79 | 116 |
| 2 | 1.0 | 1810 | 9020 | 9200 | 4.41 | 80.5 |
| 3 | 0.50 | 2300 | 13300 | 14700 | 7.51 | 116 |
| 4 | 1.0 | 2370 | 12400 | 12700 | 4.33 | 143 |
| N | 4 | 4 | 4 | 4 | 4 | 4 |
| Mean | 0.88 | 2250 | 12200 | 13000 | 5.51 | 114 |
| SD | 0.25 | 304 | 2280 | 2740 | 1.49 | 25.8 |
| CV % | 28.6 | 13.5 | 18.6 | 21.1 | 27.1 | 22.6 |

^{b}To calculate % F, 4.66 mg/kg dose was used. % F is an estimated value which is calculated from nominal ODT dose concentrations and estimated AUC_{inf} values.
Note:
Clearance following PO dosing is CL/F;
Volume following PO dosing is V_{Z}/F;
NA = Not applicable;
NC = Not calculated

Summary of mean pharmacokinetic parameters data are shown Table 20.

TABLE 20
Summary of Mean Pharmacokinetic Parameters
Following a Single Oral Disintegrating Tablet
Dose of Compound A to Male Beagle Dogs

| Cₘₐₓ | AUC₀₋₂₄ | AUC_{inf} | t_{1/2} | Percent F |
|---|---|---|---|---|
| (ng/mL) | (hr*ng/mL) | (hr*ng/mL) | (hr) | (%)^{b} |
| 2250 | 12200 | 13000 | 5.51 | 114 |

^{b}To calculate % F, 4.66 mg/kg dose was used. % F is an estimated value which is calculated from nominal ODT dose concentrations and estimated AUC_{inf} values.
Note:
Clearance following PO dosing is CL/F; Volume following PO dosing is Vz/F, respectively;
NA = Not applicable

Administration of 50.75 mg/dose Compound A via ODT was well tolerated in male Beagle dogs. Following an ODT dose of Compound A to male Beagle dogs, Compound A was quantifiable in plasma throughout the sampling period postdose (from 0.25 to 24 hours postdose). Mean peak (Cₘₐₓ) and total AUC₀₋₂₄ exposures to Compound A, half-life (t_{1/2}), and percent bioavailability (% F) were 2250 ng/mL, and 12200 hr*ng/mL, 5.51 hours, and 114% respectively.

### Example 5—Freeze/Thaw

The purpose of this thermal cycling study on Compound A is to provide data to assess impact of the freeze-thaw cycling on the product within the primary packaging to provide supporting data for shipping. The samples were cycled three times between storage at stability conditions −20° C. and 40° C./75% RH.

Testing was conducted at the completion of the freeze-thaw cycling study and the results (except for packaging appearance) were compared to the initial stability results.

After three cycles of −20° C. to 40° C./75°/0% RH storage there was no notable change in the integrity of the blister packaging. The physical and chemical stability results for the thermal cycling samples were within specification and were not significantly different from the batch release testing.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A pharmaceutical composition, comprising:
(a) compound A: 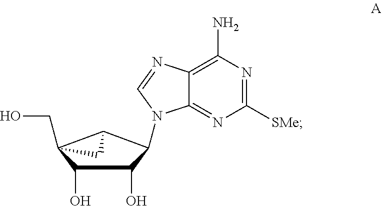
(b) A matrix former;
(c) A structure former; and, optionally,
(d) A flavoring agent and/or sweetening agent;
wherein the composition disintegrates in water at 20° C. in about 30 seconds or less.

2. The composition of claim 1, wherein the structure former comprises mannitol, PEG 400, hydrolyzed dextrose, dextran, dextrin, maltodextrin, an alginate, hydroxyethylcellulose, carboxymethylcellulose, microcrystalline cellulose, com-syrup solids, pectin, carrageenan, agar, chitosan, locust bean gum, xanthan gum, guar gum, acacia gum, tragacanth, konjac flour, rice flour, wheat gluten, sodium starch glycolate, soy fiber protein, potato protein, papain, glycine, sorbitol, erythritol, lactose, D-glucitol, trehalose, xylitol, or maltose; or a combination of two or more thereof.

3. The composition of claim 1 or 2, wherein the structure former comprises mannitol.

4. The composition of claim 3, wherein the mannitol is present as about 2.0% to about 60.0% w/w of the composition.

5. The composition of claim 3, wherein the mannitol is present as about 10.0% to 24.0% w/w of the composition.

6. The composition of claim 3, wherein the mannitol is present as about 16.0% w/w of the composition.

7. The composition of any one of claims 1-6, wherein the matrix former comprises a water-soluble or water-dispersible pharmaceutically acceptable carrier.

8. The composition of any one of claims 1-7, wherein the matrix former comprises gelatin, pullulan, starch, or combinations thereof.

9. The composition of any one of claims 1-8, wherein the matrix former comprises fish gelatin, bovine gelatin, porcine gelatin, or combinations thereof.

10. The composition of claim 9, wherein the gelatin comprises Gel NonOx (Gelita), HMW (High Molecular Weight) bovine or HMW fish gelatin.

11. The composition of claim 10, wherein the gelatin comprises HMW fish gelatin.

12. The composition of any one of claims 1-11, wherein the matrix former is present in an about 5:1 w/w to about 1:5 w/w ratio with the structure former.

13. The composition of any one of claims 1-11, wherein the matrix former is present in an about 1:1.25 w/w ratio with the structure former.

14. The composition of any one of claims 1-11, wherein the matrix former is present as about 2.0% to about 60.0% w/w of the composition.

15. The composition of any one of claims 1-11, wherein the matrix former is present as about 10.0% to about 40.0% w/w of the composition.

16. The composition of any one of claims 1-11, wherein the matrix former is present as about 14.0% to about 24.0% w/w of the composition.

17. The composition of any one of claims 1-16, wherein compound A is present as about 4% to about 90% w/w of the composition.

18. The composition of any one of claims 1-16, wherein compound A is about 20% to about 80% w/w of the composition.

19. The composition of any one of claims 1-16, wherein compound A is about 52% w/w of the composition.

20. The composition of any one of claims 1-19, wherein compound A is in the form of a hemi-hydrate.

21. The composition of any one of claims 1-20, wherein compound A comprises Form A having peaks in its XRPD at 8.0±0.2, 13.1±0.2, 16.2±0.2, 16.7±0.2, and 17.9±0.2 degree 2-theta.

22. The composition of any one of claims 1-21, wherein compound A is provided as a solid having D₉₀ of less than 30 μm prior to inclusion in the composition.

23. The composition of any one of claims 1-22, wherein the composition contains no more than 0.8% by HPLC, as compared to the AUC (area under the curve) of compound A, of the following compound:

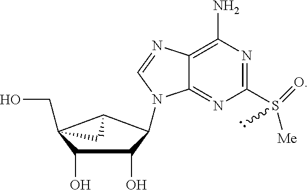

24. The composition of any one of claims 1-23, wherein the flavoring agent is present and is selected from coconut, orange, cherry, fruit punch, lemon, lime, grapefruit, raspberry, tutti-frutti, black cherry, strawberry, mint, peppermint, and combinations thereof; and the sweetening agent is present and is selected from sucrose, glucose, sucralose, fructose, lactose, aspartame, invert sugar, corn syrup, stevia extract powder, stevioside, steviol, saccharin, saccharin salts, potassium acetosulfam, sorbitol, xylitol, mannitol, erythritol, lactitol, alitame, miraculin, monellin, and thaumatin or a combination of the same.

25. The composition of claim 24, comprising about 0.10% w/w sucralose and about 0.2% w/w peppermint.

26. The composition of claim 1, comprising:
(a) compound A: 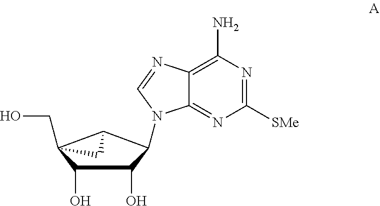
as about 4.0% to about 90.0% w/w of the composition;
(b) A matrix former comprising about 10.0% to about 40.0% w/w gelatin;
(c) A structure former comprising about 10% to about 24.0% mannitol; and, optionally,
(d) A flavoring agent and/or sweetening agent;
wherein the composition disintegrates in water at 20° C. in about 30 seconds or less.

27. The composition of claim 26, wherein compound A is present as about 40.0% to about 80% w/w of the composition; the matrix former comprises about 16.0% to about 24.0% w/w gelatin; and the structure former comprises about 12.0% to about 20.0% w/w mannitol.

28. The composition of claim 26, wherein compound A is present as about 52.0% w/w of the composition; the matrix former comprises about 20.0% w/w HMW fish gelatin; and the structure former comprises about 16.0% w/w mannitol.

29. The composition of any one of claims 1-28, wherein the composition disintegrates in water at 20° C. in about 12 seconds or less.

30. The composition of any one of claims 1-28, wherein the composition disintegrates in water at 20° C. in about 7 seconds or less.

31. The composition of any one of claims 1-28, wherein the composition disintegrates in water at 20° C. in about 0.5 to about 5 seconds.

32. A unit dosage form comprising the composition of any one of claims 1-31, wherein the unit dosage form contains about 15 mg to about 1800 mg of compound A.

33. The unit dosage form of claim 27, containing about 25 mg to about 1200 mg of compound A.

34. The unit dosage form of claim 27, containing about 50 mg, about 150 mg, about 300 mg, or about 900 mg of compound A.

35. The unit dosage form of any one of claims 32-34, wherein the unit dosage form is an oral disintegrating tablet (ODT), granules, minitablet, mucoadhesive tablet, sublingual tablet, chewable tablet, or effervescent tablet.

36. The unit dosage form of any one of claims 32-34, wherein the unit dosage form is an oral disintegrating tablet (ODT).

37. The unit dosage for of any one of claims 32-36, wherein the unit dosage forms comprises compound A in the form of a hemi-hydrate.

38. A method of preparing a pharmaceutical composition of any one of claims 1-28, comprising:
(i) mixing water, a matrix former, and a structure former to form a pre-mix;
(ii) optionally, modifying the pH of the pre-mix to a desired pH value;
(iii) optionally, heating the pre-mix to a temperature of about 20-70° C.;
(iv) optionally, adding a flavoring agent:
(v) adding compound A: 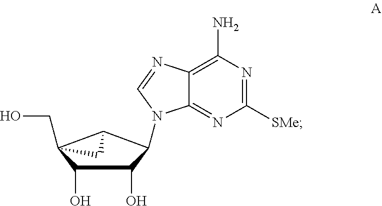
(vi) agitating the mix until compound A is dissolved, if compound A is not fully dissolved:
(vii) optionally, heating or cooling the mix of step (vi) to a temperature between about 10° C. and about 40° C.; and
(viii) removing the water and any volatile components.

39. A method of preparing an oral disintegrating tablet (ODT) of claim 36, comprising:
(i) dosing a pharmaceutical composition into a preformed mold, wherein the pharmaceutical composition comprises a matrix former, a structure former, compound A: 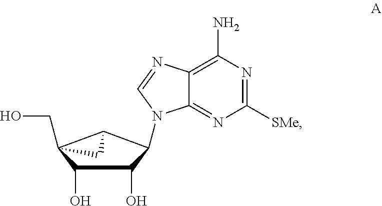
and, optionally, a flavoring agent and/or sweetening agent;
(ii) freezing the pharmaceutical composition; and
(iii) freeze-drying the pharmaceutical composition to form the ODT.

40. The method of claim 38, wherein compound A is provided in step (v) in the form of a hemi-hydrate.

41. The method of claim 40, wherein compound A comprises Form A having peaks in its XRPD at 8.0±0.2, 13.1±0.2, 16.2±0.2, 16.7±0.2, and 17.9±0.2 degree 2-theta.

42. The method of claim 38, wherein compound A is provided in anhydrous form.

43. A method of treating an injury, disease, or condition selected from traumatic brain injury (TBI), concussion, stroke, polytrauma, cardiac arrest, near drowning, altitude sickness, brain injuries from directed energy or Havanna Syndrome, partial or total spinal cord transection, malnutrition, toxic neuropathies, meningoencephalopathies, neurodegeneration caused by a genetic disorder, age-related neurodegeneration, vascular disease, Alzheimer's Disease (AD), Parkinson's Disease (PD), Huntington's Disease (HD), Multiple Sclerosis (MS), amyotrophic lateral sclerosis (ALS), chronic traumatic encephalopathy (CTE), cardiovascular disease, autoimmune diseases, allergic diseases, transplant rejection, graft-versus-host disease, intraocular hypertension, glaucoma, odor sensitivity, an olfactory disorder, type 2 diabetes, pain control, respiratory diseases, deficits in CNS function, deficits in learning, deficits in cognition, otic disorders, Meniere's disease, endolymphatic hydrops, progressive hearing loss, noise-induced hearing loss, dizziness, vertigo, tinnitus, collateral brain damage associated with radiation cancer therapy, migraine treatment, sleep disorders in the elderly, epilepsy, schizophrenia, symptoms experienced by recovering alcoholics, damage to neurons or nerves of the peripheral nervous system during surgery, gastrointestinal conditions, pain mediated by the CNS, migraine, collateral brain damage associated with radiation cancer therapy, depression, mood or behavioral changes, dementia, erratic behavior, suicidality, tremors, Huntington's chorea, loss of coordination of movement, deafness, impaired speech, dry eyes, hypomimia, attention deficit, memory loss, cognitive difficulties, vertigo, dysarthria, dysphagia, ocular abnormalities or disorientation, and addiction;
comprising administering to a patient in need thereof an effective amount of a composition of any one of claims 1-26.

44. The method of claim 43, wherein the injury, disease, or condition is selected from acute pain, chronic pain, nociceptive pain, neuropathic pain, inflammatory pain, and acute pain.

45. The method of claim 43, wherein the pain is selected from musculoskeletal pain, fibromyalgia, myofascial pain, pain during menstruation, pain during osteoarthritis, pain during rheumatoid arthritis, pain during gastrointestinal inflammation, pain during inflammation of the heart muscle, pain during multiple sclerosis, pain during neuritis, pain during AIDS, pain during chemotherapy, tumor pain, headache, chronic pain syndrome (CPS), central pain, trigeminal neuralgia, shingles, stamp pain, phantom limb pain, temporomandibular joint disorder, nerve injury, migraine, post-herpetic neuralgia, neuropathic pain encountered as a consequence of injuries, amputation infections, metabolic disorders or degenerative diseases of the nervous system, neuropathic pain associated with diabetes, pseudesthesia, hypothyroidism, uremia, vitamin deficiencies or alcoholism, acute pain after injuries, postoperative pain, pain during acute gout, and pain from an operation.

46. The method of claim 43, wherein the injury, disease, or condition is selected from traumatic brain injury (TBI), stroke, a neurodegenerative condition, and a heart and cardiovascular disease.

47. The method of claim 46, wherein the injury, disease, or condition is TBI selected from concussion, blast injury, combat-related injury, a mild, moderate or severe blow to the head, whiplash, sports-related injury, or a head injury sustained from a fall or other accident.

48. The method of claim 46, wherein the injury, disease, or condition is a stroke selected from ischemic stroke, hemorrhagic stroke, subarachnoid hemorrhage, cerebral vasospasm, and transient ischemic attacks (TIA).

49. The method of any one of claims 43-48, wherein neuroprotection or neurorestoration is increased in the patient as compared with an untreated patient.

50. The method of claim 43, wherein the injury, disease, or condition is a neurodegenerative disease selected from Alzheimer's Disease (AD), Parkinson's Disease (PD), Huntington's Disease (HD), Multiple Sclerosis (MS), amyotrophic lateral sclerosis (ALS), chronic traumatic encephalopathy (CTE), and a neurodegenerative condition caused by a virus, alcoholism, tumor, toxin, or repetitive brain injuries.

51. The method of claim 43, wherein the injury, disease, or condition is heart or cardiovascular disease selected from cardiac ischemia, myocardial infarction, a cardiomyopathy, coronary artery disease, arrhythmia, myocarditis, pericarditis, angina, hypertensive heart disease, endocarditis, rheumatic heart disease, congenital heart disease, and atherosclerosis.

52. A method of increasing neuroprotection or neurorestoration in a patient in need thereof who has suffered a TBI or stroke, comprising administering to the patient an effective amount of the composition of any one of claims 1-26.

53. A method of treating an injury, disease, disorder, or condition selected from:
(i) brain damage caused by radiation or collateral brain damage associated with radiation cancer therapy or migraine treatment;
(ii) migraine headache;
(iii) a condition associated with a brain injury or a neurodegenerative condition; and
(iv) an autoimmune disease or condition, glaucoma, an otic disorder, progressive hearing loss, tinnitus, epilepsy, pain control, pain mediated by the CNS, neuropathic pain, inflammatory pain, or acute pain:
comprising administering to a patient in need thereof an effective amount of the composition of any one of claims 1-26.

54. The method of claim 53, wherein the composition increases neuroprotection or neurorestoration in the patient as compared with an untreated patient.

55. The method of claim 53, wherein the condition associated with a brain injury or a neurodegenerative condition is selected from epilepsy, migraine, collateral brain damage associated with radiation cancer therapy, depression, mood or behavioral changes, dementia, erratic behavior, suicidality, tremors, Huntington's chorea, loss of coordination of movement, deafness, impaired speech, dry eyes, hypomimia, attention deficit, memory loss, cognitive difficulties or deficit in cognition, deficit in CNS function, deficit in learning, vertigo, dysarthria, dysphagia, ocular abnormalities, and disorientation.

56. The method according to claim 53, wherein the injury, disease, or condition is migraine.

57. The method according to claim 53, wherein the injury, disease, or condition is pain selected from central pain syndrome, peripheral neuropathy, corneal neuropathic pain, post stroke pain, and pain caused by multiple sclerosis.

58. A method of increasing cardioprotection or regeneration of damaged heart tissue in a patient in need thereof who has suffered a cardiac ischemia or myocardial infarction, comprising administering to the patient an effective amount of the composition of any one of claims 1-26.

59. A method of treating an addiction, addictive behavior, behavioral addiction, brain reward system disorder, or a compulsive disorder, comprising administering to a patient in need thereof an effective amount of the composition of any one of claims 1-26.

60. The method of any one of claims 43-59, wherein the composition of any one of claims 1-26 is administered once an hour, every hour, for 1 to 8 hours.

61. The method of any one of claims 43-59, wherein the composition of any one of claims 1-26 is administered once an hour or once every two hours.

62. The method of any one of claims 43-59, wherein the composition of any one of claims 1-26 is administered up to three times over five hours.
